(19) United States
(12) Patent Application Publication
(10) Pub. No.: US 2009/0298800 A1
(43) Pub. Date: Dec. 3, 2009

(54) 1,25-DIHYDROXY, 20-CYCLOPROPYL,26-27-DEUTEROALKYL VITAMIN D3 COMPOUNDS AND METHODS OF USE THEREOF

(75) Inventors: Milan R. Uskokovic, Upper Montclair, NJ (US); Stanislaw Marczak, Wayne, NJ (US); Pawel Jankowski, Wayne, NJ (US); Luciano Adorini, Milan (IT)

Correspondence Address: EDWARDS ANGELL PALMER & DODGE LLP, P.O. BOX 55874, BOSTON, MA, 02205, US

(73) Assignee: BioXell S.p.A., Milan (IT)

(21) Appl. No.: 12/067,873

(22) PCT Filed: Sep. 21, 2006
(86) PCT No.: PCT/US06/36944

§ 371 Date: Nov. 24, 2008

Related U.S. Application Data
(60) Provisional application No. 60/720,234, filed on Sep. 23, 2005.
(60) Provisional application No. 60/729,438, filed on Oct. 21, 2005.

(51) Int. Cl. A61K 31/59; C07C 401/00; A61P 37/00; A61P 35/00

(52) U.S. Cl. 514/167; 552/653

(57) ABSTRACT

The invention provides vitamin D₃ analogs of cholecalciferol, substituted at carbons-26 and 27 with deuterated alkyl groups, e.g., trideuteromethyl, wherein carbon-16 is a single or double bond, and carbon-23 is a single, double, or triple bond. The invention provides pharmaceutically acceptable esters, salts, and prodrugs thereof. Methods for using the compounds to treat vitamin D₃ associated states, and pharmaceutical compositions containing the compounds are also disclosed.

### RELATED APPLICATIONS

This application claims the benefit of U.S. provisional patent application Ser. No. 60/720,234, filed Sep. 23, 2005, and U.S. provisional patent application Ser. No. 60/729,438, filed Oct. 21, 2005. The disclosures of the aforementioned provisional patent applications are incorporated herein in their entireties by this reference.

### BACKGROUND OF THE INVENTION

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” that was essential for the normal development of the skeleton and maintenance of calcium and phosphorous homeostasis.

Studies involving the metabolism of vitamin D₃ were initiated with the discovery and chemical characterization of the plasma metabolite, 25-hydroxyvitamin D₃ [25(OH)D₃] (Blunt, J. W. et al (1968)Biochemistry 6:3317-3322) and the hormonally active form, 1α,25(OH)₂D₃ (Myrtle, J. F. et al. (1970)J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad. Sci. USA 68:803-804). The formulation of the concept of a vitamin D endocrine system was dependent both upon appreciation of the key role of the kidney in producing 1α, 25 (OH)₂D₃ in a carefully regulated fashion (Fraser, D. R. and Kodicek, E (1970) Nature 288:764-766; Wong, R. G. et al. (1972)J. Clin. Invest. 51:1287-1291), and the discovery of a nuclear receptor for 1 α,25(OH)₂D₃ (VD₃R) in the intestine (Haussler, M. R. et al. (1969) Exp. Cell Res. 58:234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427-432; Ohyama, Y. and Okuda, K. (1991) J. Biol. Chem. 266:8690-8695) and kidney (Henry, H. L. and Norman, A. W. (1974) J. Biol. Chem. 249:7529-7535; Gray, R. W. and Ghazarian, J. G. (1989) Biochem. J. 259:561-568), and in a variety of other tissues to effect the conversion of vitamin D₃ into biologically active metabolites such as 1α, 25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N.E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1α,25(OH)₂D₃ to generate the requisite specific biological responses for this secosteroid hormone (Pike, J. W. (1991) Annu. Rev. Nutr. 11: 189-216). To date, there is evidence that nuclear receptors for 1α,25(OH)₂D₃ (VD₃R) exist in more than 30 tissues and cancer cell lines (Reichel, H. and Norman, A. W. (1989) Annu. Rev. Med. 40:71-78).

Vitamin D₃ and its hormonally active forms are well-known regulators of calcium and phosphorous homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis. A paracrine role for 1α,25(OH)₂ D₃ has been suggested by the combined presence of enzymes capable of oxidizing vitamin D₃ into its active forms, e.g., 25-OHD-1α-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40: 71-78).

Given the activities of vitamin D₃ and its metabolites, much attention has focused on the development of synthetic analogs of these compounds. A large number of these analogs involve structural modifications in the A ring, B ring, C/D rings, and, primarily, the side chain (Bouillon, R. et al., Endocrine Reviews 16(2):201-204). Although a vast majority of the vitamin D₃ analogs developed to date involve structural modifications in the side chain, a few studies have reported the biological profile of A-ring diastereomers (Norman, A. W. et al. J. Biol. Chem. 268 (27): 20022-20030). Furthermore, biological esterification of steroids has been studied (Hochberg, R. B., (1998) Endocr Rev. 19(3): 331-348), and esters of vitamin D₃ are known (WO 97/11053).

Moreover, despite much effort in developing synthetic analogs, clinical applications of vitamin D and its structural analogs have been limited by the undesired side effects elicited by these compounds after administration to a subject for known indications/applications of vitamin D compounds.

### SUMMARY OF THE INVENTION

In one aspect, the invention provides a vitamin D₃ compound of formula I:

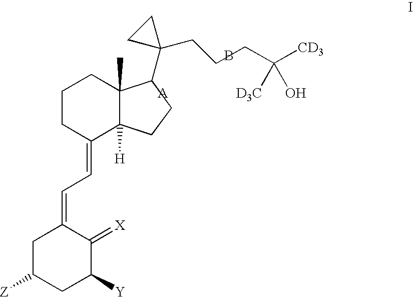

wherein: A is single or double bond; B is a single, double, or triple bond; X is H₂ or CH₂; Y is hydroxyl, OC(O)C₁-C₄ alkyl, OC(O)hydroxyalkyl, OC(O)haloalkyl; or halogen; Z is hydroxyl, OC(O)C₁-C₄ alkyl, OC(O)hydroxyalkyl, or OC(O)haloalkyl; and pharmaceutically acceptable esters, salts, and prodrugs thereof.

In yet another aspect, the invention provides a pharmaceutical composition. The composition comprises an effective amount of a vitamin D₃ compound of formula I, and a pharmaceutically acceptable carrier.

In a further aspect, the method provides a method of ameliorating a deregulation of calcium and phosphate metabolism. The method includes administering to a subject a therapeutically effective amount of a vitamin D₃ compound of formula I, so as to ameliorate the deregulation of the calcium and phosphate metabolism.

In another aspect, the invention provides a method of modulating the expression of an immunoglobulin-like transcript 3 (ILT3) surface molecule in a cell. The method includes contacting the cell with a vitamin D₃ compound of formula I in an amount effective to modulate the expression of an immunoglobulin-like transcript 3 (ILT3) surface molecule in the cell.

In yet another aspect, the invention provides a method of treating an ILT3-associated disorder in a subject. The method includes administering to the subject a vitamin D₃ compound of formula I in an amount effective to modulate the expression of an ILT3 surface molecule, thereby treating the ILT3-associated disorder in the subject.

In still another aspect, the invention provides a method of inducing immunological tolerance in a subject. The method includes administering to the subject a vitamin D₃ compound of formula I in an amount effective to modulate the expression of an ILT3 surface molecule, thereby inducing immunological tolerance in the subject.

In a further aspect, the invention provides a method of inhibiting transplant rejection in a subject. The method includes administering to the subject a vitamin D₃ compound of formula I in an amount effective to modulate the expression of an ILT3 surface molecule, thereby inhibiting transplant rejection in the subject.

In still another embodiment, the invention provides a method for preventing or treating bladder dysfunction in a subject in need thereof by administering an effective amount of a vitamin D₃ compound of formula I thereby to prevent or treat bladder dysfunction in said subject.

In yet another aspect, the invention provides a packaged formulation for use in the treatment of a vitamin D₃ associated state. The packaged formulation includes a pharmaceutical composition comprising a vitamin D₃ compound of formula I and a pharmaceutically-acceptable carrier, packaged with instructions for use in the treatment of a vitamin D₃ associated state.

In another aspect, the invention provides a packaged formulation for use in the treatment of an ILT-3 associated disorder. The packed formulation includes a pharmaceutical composition comprising a vitamin D₃ compound of formula I and a pharmaceutically-acceptable carrier, packaged with instructions for use in the treatment of an ILT3-associated disorder.

In a further aspect, the invention provides a method for modulating immunosuppressive activity by an antigen-presenting cell. The method includes contacting an antigen-presenting cell with a vitamin D₃ compound of formula I in an amount effective to modulate ILT3 surface molecule expression, thereby modulating the immunosuppressive activity by the antigen-presenting cell.

### DETAILED DESCRIPTION OF THE INVENTION

### 1. Definitions

Before further description of the present invention, and in order that the invention may be more readily understood, certain terms are first defined and collected here for convenience.

The term “administration” or “administering” includes routes of introducing the vitamin D₃ compound(s) to a subject to perform their intended function. Examples of routes of administration which can be used include injection (subcutaneous, intravenous, parenterally, intraperitoneally, intrathecal), oral, inhalation, rectal and transdermal. The pharmaceutical preparations are, of course, given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the vitamin D₃ compound can be coated with or disposed in a selected material to protect it from natural conditions which may detrimentally effect its ability to perform its intended function. The vitamin D₃ compound can be administered alone, or in conjunction with either another agent as described above or with a pharmaceutically-acceptable carrier, or both. The vitamin D₃ compound can be administered prior to the administration of the other agent, simultaneously with the agent, or after the administration of the agent. Furthermore, the vitamin D₃ compound can also be administered in a proform which is converted into its active metabolite, or more active metabolite in vivo.

The term “alkyl” refers to the radical of saturated aliphatic groups, including straight-chain alkyl groups, branched-chain alkyl groups, cycloalkyl(alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer, and still more preferably 4 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “antigen” includes a substance which elicits an immune response. The antigens of the invention to which tolerance is induced may or may not be exogenously derived relative to the host. For example, the method of the invention may be used to induce tolerance to an “autoantigen.” An autoantigen is a normal constituent of the body that reacts with an autoantibody. The invention also includes inducing tolerance to an “alloantigen.” Alloantigen refers to an antigen found only in some members of a species, for example the blood group substances. An allograft is a graft to a genetically different member of the same species. Allografts are rejected by virtue of the immunological response of T lymphocytes to histocompatibility antigens. The method of the invention also provides for inducing tolerance to a “xenoantigen.” Xenoantigens are substances that cause an immune reaction due to differences between different species. Thus, a xenograft is a graft from a member of one species to a member of a different species. Xenografts are usually rejected within a few days by antibodies and cytotoxic T lymphocytes to histocompatibility antigens.

The language “antigen-presenting cell” or “APC” includes a cell that is able to present an antigen to, for example, a T helper cell. Antigen-presenting cells include B lymphocytes, accessory cells or non-lymphocytic cells, such as dendritic cells, Langerhans cells, and mononuclear phagocytes that help in the induction of an immune response by presenting antigen to helper T lymphocytes. The antigen-presenting cell of the present invention is preferably of myeloid origin, and includes, but is not limited to, dendritic cells, macrophages, monocytes. APCs of the present invention may be isolated from the bone marrow, blood, thymus, epidermis, liver, fetal liver, or the spleen.

The terms “antineoplastic agent” and “antiproliferative agent” are used interchangeably herein and includes agents that have the functional property of inhibiting the proliferation of a vitamin D₃-responsive cells, e.g., inhibit the development or progression of a neoplasm having such a characteristic, particularly a hematopoietic neoplasm.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The language “autoimmune disease” or “autoimmune disorder” refers to the condition where the immune system attacks the host's own tissue(s). In an autoimmune disease, the immune tolerance system of the patient fails to recognize self antigens and, as a consequence of this loss of tolerance, brings the force of the immune system to bear on tissues which express the antigen. Autoimmune disorders include, but are not limited to, type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitis, uveoretinitis, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease.

The language “biological activities” of vitamin D₃ includes all activities elicited by vitamin D₃ compounds in a responsive cell. It includes genomic and non-genomic activities elicited by these compounds (Gniadecki R. and Calverley M. J. (1998) Pharmacology & Toxicology 82: 173-176; Bouillon, R. et al. (1995) Endocrinology Reviews 16(2):206-207; Norman A. W. et al. (1992) J. Steroid Biochem Mol. Biol. 41:231-240; Baran D. T. et al. (1991)J. Bone Miner Res. 6:1269-1275; Caffrey J. M. and Farach-Carson M. C. (1989) J. Biol. Chem. 264:20265-20274; Nemere I. et al. (1984) Endocrinology 115:1476-1483).

By “bladder dysfunction” is meant bladder conditions associated with overactivity of the detrusor muscle, for example, clinical BPH or overactive bladder. In the context of the present invention “bladder dysfunction” excludes bladder cancer.

The language “bone metabolism” includes direct or indirect effects in the formation or degeneration of bone structures, e.g., bone formation, bone resorption, etc., which may ultimately affect the concentrations in serum of calcium and phosphate. This term is also intended to include effects of compounds of the invention in bone cells, e.g., osteoclasts and osteoblasts, that may in turn result in bone formation and degeneration.

The language “calcium and phosphate homeostasis” refers to the careful balance of calcium and phosphate concentrations, intracellularly and extracellularly, triggered by fluctuations in the calcium and phosphate concentration in a cell, a tissue, an organ or a system. Fluctuations in calcium levels that result from direct or indirect responses to compounds of the invention are intended to be included by these terms.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, bladder, breast, head and neck, colon and ovary. The term also includes carcinosarcomas, e.g., which include malignant tumors composed of carcinomatous and sarcomatous tissues.

An “adenocarcinoma” refers to a carcinoma derived from glandular tissue or in which the tumor cells form recognizable glandular structures.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “effective amount” includes an amount effective, at dosages and for periods of time necessary, to achieve the desired result, e.g., sufficient treat a vitamin D₃ associated state or to modulate ILT3 expression in a cell. An effective amount of vitamin D₃ compound may vary according to factors such as the disease state, age, and weight of the subject, and the ability of the vitamin D₃ compound to elicit a desired response in the subject. Dosage regimens may be adjusted to provide the optimum therapeutic response. An effective amount is also one in which any toxic or detrimental effects (e.g., side effects) of the vitamin D₃ compound are outweighed by the therapeutically beneficial effects.

A therapeutically effective amount of vitamin D₃ compound (i.e., an effective dosage) may range from about 0.001 to 30 μg/kg body weight, preferably about 0.01 to 25 μg/kg body weight, more preferably about 0.1 to 20 μg/kg body weight, and even more preferably about 1 to 10 μg/kg, 2 to 9 μg/kg, 3 to 8 μg/kg, 4 to 7 μg/kg, or 5 to 6 μg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a vitamin D₃ compound can include a single treatment or, preferably, can include a series of treatments. In one example, a subject is treated with a vitamin D₃ compound in the range of between about 0.1 to 20 μg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a vitamin D₃ compound used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The language “genomic” activities or effects of vitamin D₃ is intended to include those activities mediated by the nuclear receptor for 1α,25(OH)₂D₃ (VD₃R), e.g., transcriptional activation of target genes.

The term “haloalkyl” is intended to include alkyl groups as defined above that are mono-, di- or polysubstituted by halogen, e.g., fluoromethyl and trifluoromethyl.

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “hormone secretion” is art-recognized and includes activities of vitamin D₃ compounds that control the transcription and processing responsible for secretion of a given hormone e.g., a parathyroid hormone (PTH) of a vitamin D₃ responsive cell (Bouillon, R. et al. (1995) Endocrine Reviews 16(2):235-237).

The language “hypercalcemia” or “hypercalcemic activity” is intended to have its accepted clinical meaning, namely, increases in calcium serum levels that are manifested in a subject by the following side effects, depression of central and peripheral nervous system, muscular weakness, constipation, abdominal pain, lack of appetite and, depressed relaxation of the heart during diastole. Symptomatic manifestations of hypercalcemia are triggered by a stimulation of at least one of the following activities, intestinal calcium transport, bone calcium metabolism and osteocalcin synthesis (reviewed in Boullion, R. et al. (1995) Endocrinology Reviews 16(2): 200-257).

The terms “hyperproliferative” and “neoplastic” are used interchangeably, and include those cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The language “immunoglobulin-like transcript 3” or “ILT3” refers to a cell surface molecule of the immunoglobulin superfamily, which is expressed by antigen-presenting cells (APCs) such as monocytes, macrophages and dendritic cells. ILT3 is a member of the immunoglobulin-like transcript (ILT) family and displays a long cytoplasmic tail containing putative immunoreceptor tyrosine-based inhibitory motifs (ITIMs). ILT3 has been shown to behave as an inhibitory receptor when cross-linked to a stimulatory receptor. A cytoplasmic component of the ILT3-mediated signaling pathway is the SH2-containing phosphatase SHP-1, which becomes associated with ILT3 upon cross-linking. ILT3 is also internalized and ILT3 ligands are efficiently presented to specific T cells (see, e.g., Cella, M. et al. (1997) J. Exp. Med. 185:1743). The determination of whether the candidate vitamin D₃ compound modulates the expression of the ILT3 surface molecule can be accomplished, for example, by comparison of ILT3 surface molecule expression to a control, by measuring mRNA expression, or by measuring protein expression.

An “ILT3-associated disorder” includes a disease, disorder or condition which is associated with an ILT3 molecule. ILT3 associated disorders include disorders in which ILT3 activity is aberrant or in which a non-ILT3 activity that would benefit from modulation of an ILT3 activity is aberrant. In one embodiment, the ILT3-associated disorder is an immune disorder, e.g., an autoimmune disorder, such as type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitis, uveoretinitis, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease; or transplant rejection, such as GVHD. In certain embodiments of the invention, the ILT3 associated disorder is an immune disorders, such as transplant rejections, graft versus host disease and autoimmune disorders.

The term “immune response” includes T and/or B cell responses, e.g., cellular and/or humoral immune responses. The claimed methods can be used to reduce both primary and secondary immune responses. The immune response of a subject can be determined by, for example, assaying antibody production, immune cell proliferation, the release of cytokines, the expression of cell surface markers, cytotoxicity, and the like.

The terms “immunological tolerance” or “tolerance to an antigen” or “immune tolerance” include unresponsiveness to an antigen without the induction of a prolonged generalized immune deficiency. Consequently, according to the invention, a tolerant host is capable of reacting to antigens other than the tolerizing antigen. Tolerance represents an induced depression in the response of a subject that, had it not been subjected to the tolerance-inducing procedure, would be competent to mount an immune response to that antigen. In one embodiment of the invention, immunological tolerance is induced in an antigen-presenting cell, e.g., an antigen-presenting cell derived from the myeloid or lymphoid lineage, dendritic cells, monocytes and macrophages.

The language “immunosuppressive activity” refers to the process of inhibiting a normal immune response. Included in this response is when T and/or B clones of lymphocytes are depleted in size or suppressed in their reactivity, expansion or differentiation. Immunosuppressive activity may be in the form of inhibiting or blocking an immune response already in progress or may involve preventing the induction of an immune response. The functions of activated T cells may be inhibited by suppressing immune cell responses or by inducing specific tolerance, or both. Immunosuppression of T cell responses is generally an active, non-antigen-specific, process that requires continuous exposure of the T cells to the suppressive agent. Tolerance, which involves inducing non-responsiveness or energy in T cells, is distinguishable from immunosuppression in that it is generally antigen-specific and persists after exposure to the tolerizing agent has ceased. Operationally, tolerance can be demonstrated by the lack of a T cell response upon re-exposure to specific antigen in the absence of the tolerizing agent.

The language “improved biological properties” refers to any activity inherent in a compound of the invention that enhances its effectiveness in vivo. In a preferred embodiment, this term refers to any qualitative or quantitative improved therapeutic property of a vitamin D₃ compound, such as reduced toxicity, e.g., reduced hypercalcemic activity.

The language “inhibiting the growth” of the neoplasm includes the slowing, interrupting, arresting or stopping its growth and metastases and does not necessarily indicate a total elimination of the neoplastic growth.

The phrase “inhibition of an immune response” is intended to include decreases in T cell proliferation and activity, e.g., a decrease in IL₂, interferon-γ, GM-CSF synthesis and secretion (Lemire, J. M. (1992) J. Cell Biochemistry 49:26-31, Lemire, J. M. et al. (1994) Endocrinology 135 (6): 2813-2821; Bouillon, R. et al. (1995) Endocrine Review 16 (2):231-32).

The term “isomers” or “stereoisomers” refers to compounds which have identical chemical constitution, but differ with regard to the arrangement of the atoms or groups in space.

The term “leukemia” is intended to have its clinical meaning, namely, a neoplastic disease in which white corpuscle maturation is arrested at a primitive stage of cell development. The disease is characterized by an increased number of leukemic blast cells in the bone marrow, and by varying degrees of failure to produce normal hematopoietic cells. The condition may be either acute or chronic. Leukemia's are further typically categorized as being either lymphocytic i.e., being characterized by cells which have properties in common with normal lymphocytes, or myelocytic (or myelogenous), i.e., characterized by cells having some characteristics of normal granulocytic cells. Acute lymphocytic leukemia (“ALL”) arises in lymphoid tissue, and ordinarily first manifests its presence in bone marrow. Acute myelocytic leukemia (“AML”) arises from bone marrow hematopoietic stem cells or their progeny. The term acute myelocytic leukemia subsumes several subtypes of leukemia: myeloblastic leukemia, promyelocytic leukemia, and myelomonocytic leukemia. In addition, leukemias with erythroid or megakaryocytic properties are considered myelogenous leukemias as well.

The term “leukemic cancer” refers to all cancers or neoplasias of the hemopoietic and immune systems (blood and lymphatic system). The acute and chronic leukemias, together with the other types of tumors of the blood, bone marrow cells (myelomas), and lymph tissue (lymphomas), cause about 10% of all cancer deaths and about 50% of all cancer deaths in children and adults less than 30 years old. Chronic myelogenous leukemia (CML), also known as chronic granulocytic leukemia (CGL), is a neoplastic disorder of the hematopoietic stem cell. The term “leukemia” is art recognized and refers to a progressive, malignant disease of the blood-forming organs, marked by distorted proliferation and development of leukocytes and their precursors in the blood and bone marrow.

The term “modulate” refers to increases or decreases in the activity of a cell in response to exposure to a compound of the invention, e.g., the inhibition of proliferation and/or induction of differentiation of at least a sub-population of cells in an animal such that a desired end result is achieved, e.g., a therapeutic result. In preferred embodiments, this phrase is intended to include hyperactive conditions that result in pathological disorders.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangeably, as their context will reveal, referring to generally to cells experiencing abnormal cell growth rates. Neoplasias and hyperplasias include “tumors,” which may be either benign, premalignant or malignant.

The language “non-genomic” vitamin D₃ activities include cellular (e.g., calcium transport across a tissue) and subcellular activities (e.g., membrane calcium transport opening of voltage-gated calcium channels, changes in intracellular second messengers) elicited by vitamin D₃ compounds in a responsive cell. Electrophysiological and biochemical techniques for detecting these activities are known in the art. An example of a particular well-studied non-genomic activity is the rapid hormonal stimulation of intestinal calcium mobilization, termed “transcaltachia” (Nemere I. et al. (1984) Endocrinology 115:1476-1483; Lieberherr M. et al. (1989)J. Biol. Chem. 264:20403-20406; Wali R. K. et al. (1992) Endocrinology 131:1125-1133; Wali R. K. et al. (1992) Am. J. Physiol. 262:G945-G953; Wali R. K. et al. (1990) J. Clin. Invest. 85:1296-1303; Bolt M. J. G. et al. (1993) Biochem. J. 292:271-276). Detailed descriptions of experimental transcaltachia are provided in Norman, A. W. (1993) Endocrinology 268(27):20022-20030; Yoshimoto, Y. and Norman, A. W. (1986) Endocrinology 118:2300-2304. Changes in calcium activity and second messenger systems are well known in the art and are extensively reviewed in Bouillion, R. et al. (1995) Endocrinology Review 16(2): 200-257; the description of which is incorporated herein by reference.

As used herein, the term “obtaining” includes purchasing, synthesizing, isolating or otherwise acquiring one or more of the vitamin D compounds used in practicing the invention.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective anti-neoplastic amount” of a compound refers to an amount of a vitamin D₃ compound of the formula (I) or otherwise described herein which is effective, upon single or multiple dose administration to the patient, in preventing or delaying the occurrence of the onset of a neoplastic disease state.

The term “psoriasis” is intended to have its medical meaning, namely, a disease which afflicts primarily the skin and produces raised, thickened, scaling, nonscarring lesions. The lesions are usually sharply demarcated erythematosus papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling.

The language “reduced toxicity” is intended to include a reduction in any undesired side effect elicited by a vitamin D₃ compound when administered in vivo, e.g., a reduction in the hypercalcemic activity.

The term “sarcoma” is art recognized and refers to malignant tumors of mesenchymal derivation.

The term “secosteroid” is art-recognized and includes compounds in which one of the cyclopentanoperhydro-phenanthrene rings of the steroid ring structure is broken. 1α,25(OH)₂D₃ and analogs thereof are hormonally active secosteroids. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1α,25(OH)₂D₃ is illustrated herein having all carbon atoms numbered using standard steroid notation.

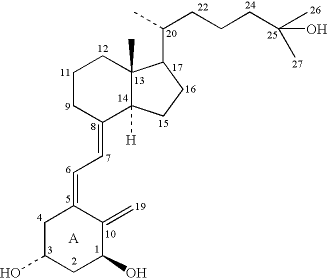

In the formulas presented herein, the various substituents on ring A are illustrated as joined to the steroid nucleus by one of these notations: a dotted line ( [symbol] ) or( [symbol] ) indicating a substituent which is in the β-orientation (i.e., above the plane of the ring), a wedged solid line ( [symbol] ) indicating a substituent which is in the α-orientation (i.e., below the plane of the molecule), or a wavy line ( [symbol] ) indicating that a substituent may be either above or below the plane of the ring. In regard to ring A, it should be understood that the stereochemical convention in the vitamin D field is opposite from the general chemical field, wherein a dotted line indicates a substituent on Ring A which is in an α-orientation (i.e., below the plane of the molecule), and a wedged solid line indicates a substituent on ring A which is in the β-orientation (i.e., above the plane of the ring). As shown, the A ring of the hormone 1α,25(OH)₂D₃ contains two asymmetric centers at carbons 1 and 3, each one containing a hydroxyl group in well-characterized configurations, namely the 1α- and 3 β-hydroxyl groups. In other words, carbons 1 and 3 of the A ring are said to be “chiral carbons” or “carbon centers.”

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. As shown, the A ring of the hormone 1-alpha,25(OH)₂D₃ contains two asymmetric centers at carbons 1 and 3, each one containing a hydroxyl group in well-characterized configurations, namely the 1-alpha- and 3-beta-hydroxyl groups. In other words, carbons 1 and 3 of the A ring are said to be “chiral carbons” or “chiral carbon centers.” Regardless, both configurations, cis/trans and/or Z/E are contemplated for the compounds for use in the present invention.

With respect to the nomenclature of a chiral center, the terms “d” and “1” configuration are as defined by the IUPAC Recommendations. As to the use of the terms, diastereomer, racemate, epimer and enantiomer, these will be used in their normal context to describe the stereochemistry of preparations.

Also, throughout the patent literature, the A ring of a vitamin D compound is often depicted in generic formulae as any one of the following structures:

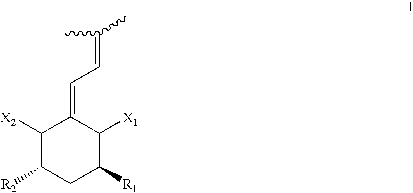

wherein X₁ and X₂ are defined as H or ═CH₂; or

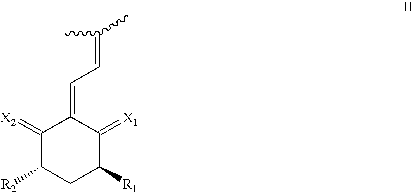

wherein X₁ and X₂ are defined as H₂ or CH₂.

Although there does not appear to be any set convention, it is clear that one of ordinary skill in the art understands either formula I or II to represent an A ring in which, for example, X₁ is ═CH₂ and X₂ is defined as H₂, as follows:

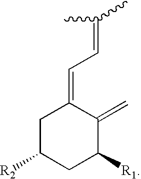

For purposes of the instant invention, formula II will be used in all generic structures.

The term “sulfhydryl” or “thiol” means —SH.

The term “subject” includes organisms which are capable of suffering from a vitamin D₃ associated state or who could otherwise benefit from the administration of a vitamin D₃ compound of the invention, such as human and non-human animals. Preferred human animals include human patients suffering from or prone to suffering from a vitamin D₃ associated state, as described herein. The term “non-human animals” of the invention includes all vertebrates, e.g., mammals, e.g., rodents, e.g., mice, and non-mammals, such as non-human primates, sheep, dog, cow, chickens, amphibians, reptiles, etc.

The phrases “systemic administration,” “administered systemically”, “peripheral administration” and “administered peripherally” as used herein mean the administration of a vitamin D₃ compound(s), drug or other material, such that it enters the patient's system and, thus, is subject to metabolism and other like processes, for example, subcutaneous administration.

The language “therapeutically effective anti-neoplastic amount” of a vitamin D₃ compound of the invention refers to an amount of an agent which is effective, upon single or multiple dose administration to the patient, in inhibiting the growth of a neoplastic vitamin D₃-responsive cells, or in prolonging the survivability of the patient with such neoplastic cells beyond that expected in the absence of such treatment.

The language “transplant rejection” refers to an immune reaction directed against a transplanted organ(s) from other human donors (allografts) or from other species such as sheep, pigs, or non-human primates (xenografts). Therefore, the method of the invention is useful for preventing an immune reaction to transplanted organs from other human donors (allografts) or from other species (xenografts). Such tissues for transplantation include, but are not limited to, heart, liver, kidney, lung, pancreas, pancreatic islets, bone marrow, brain tissue, cornea, bone, intestine, skin, and hematopoietic cells. Also included within this definition is “graft versus host disease” of “GVHD,” which is a condition where the graft cells mount an immune response against the host. Therefore, the method of the invention is useful in preventing graft versus host disease in cases of mismatched bone marrow or lymphoid tissue transplanted for the treatment of acute leukemia, aplastic anemia, and enzyme or immune deficiencies, for example. The term “transplant rejection” also includes disease symptoms characterized by loss of organ function. For example, kidney rejection would be characterized by a rising creatine level in blood. Heart rejection is characterized by an endomyocardial biopsy, while pancreas rejection is characterized by rising blood glucose levels. Liver rejection is characterized by the levels of transaminases of liver origin and bilirubin levels in blood. Intestine rejection is determined by biopsy, while lung rejection is determined by measurement of blood oxygenation.

The term “VDR” is intended to include members of the type II class of steroid/thyroid superfamily of receptors (Stunnenberg, H. G. (1993) Bio Essays 15(5):309-15), which are able to bind and transactivate through the vitamin D response element (VDRE) in the absence of a ligand (Damm et al. (1989) Nature 339:593-97; Sap et al. Nature 343:177-180).

The term “VDRE” refers to DNA sequences composed of half-sites arranged as direct repeats. It is known in the art that type II receptors do not bind to their respective binding site as homodimers but require an auxiliary factor, RXR (e.g. RXRα, RXRβ, RXRγ) for high affinity binding Yu et al. (1991) Cell 67:1251-1266; Bugge et al. (1992) EMBO J. 11:1409-1418; Kliewer et al. (1992) Nature 355:446-449; Leid et al. (1992)EMBO J. 11:1419-1435; Zhang et al. (1992) Nature 355:441-446).

The language “vitamin D₃ associated state” is a state which can be prevented, treated or otherwise ameliorated by administration of one or more compounds of the invention. Vitamin D₃ associated states include ILT3-associated disorders, disorders characterized by an aberrant activity of a vitamin D₃-responsive cell, disorders characterized by a deregulation of calcium and phosphate metabolism, and other disorders or states described herein.

The term “vitamin D₃-responsive cell” includes any cell which is capable of responding to a vitamin D₃ compound having the formula I or otherwise described herein, or is associated with disorders involving an aberrant activity of hyperproliferative skin cells, parathyroid cells, neoplastic cells, immune cells, and bone cells. These cells can respond to vitamin D₃ activation by triggering genomic and/or non-genomic responses that ultimately result in the modulation of cell proliferation, differentiation survival, and/or other cellular activities such as hormone secretion. In a preferred embodiment, the ultimate responses of a cell are inhibition of cell proliferation and/or induction of differentiation-specific genes. Exemplary vitamin D₃ responsive cells include immune cells, bone cells, neuronal cells, endocrine cells, neoplastic cells, epidermal cells, endodermal cells, smooth muscle cells, among others.

With respect to the nomenclature of a chiral center, terms “d” and “l” configuration are as defined by the IUPAC Recommendations. As to the use of the terms, diastereomer, racemate, epimer and enantiomer will be used in their normal context to describe the stereochemistry of preparations.

### 2. Vitamin D₃ Compounds of the Invention

The vitamin D₃ compounds of formula I above exert a full spectrum of 1,25(OH)₂D₃ biological activities such as binding to the specific nuclear receptor VDR, suppression of the increased parathyroid hormone levels in 5,6-nephrectomized rats, suppression of INF-γ release in MLR cells, stimulation of HL-60 leukemia cell differentiation and inhibition of solid tumor cell proliferation. It is well known that in vivo and in cellular cultures 1,25-(OH)₂D₃ undergoes a cascade of metabolic modifications initiated by the influence of 24R-hydroxylase enzyme. First 24R-hydroxy metabolite is formed, which is oxidized to 24-keto intermediate, and then 23S-hydroxylation and fragmentation produce the fully inactive calcitroic acid.

Thus, in one aspect, the invention provides a vitamin D₃ compound of formula I:

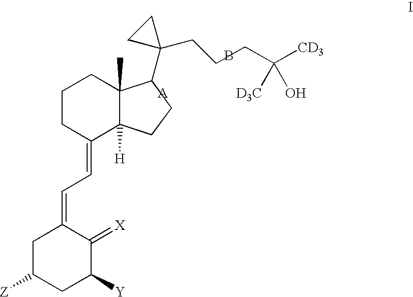

wherein: A is single or double bond; B is a single, double, or triple bond; X is H₂ or CH₂;
Y is hydroxyl, OC(O)C₁-C₄ alkyl, OC(O)hydroxyalkyl, OC(O)haloalkyl; or halogen; Z is hydroxyl, OC(O)C₁-C₄ alkyl, OC(O)hydroxyalkyl, or OC(O)haloalkyl; and pharmaceutically acceptable esters, salts, and prodrugs thereof.

In one embodiment, the invention provides a compound, wherein Y is hydroxyl. In another embodiment, Y is halogen. In a further embodiment, Y is F. In another embodiment, Y is OC(O)C₁-C₄ alkyl, preferably OC(O)CH₃. In another embodiment, Z is hydroxyl. In yet another embodiment, A is a single bond. In still another embodiment, A is a double bond. In another embodiment, B is a single bond. In still another embodiment, B is a double bond. In yet another embodiment, B is a triple bond. In one embodiment, X is CH₂. In another embodiment, X is H₂.

Preferred species of the invention include the following: 1,25-Dihydroxy-20-cyclopropyl-26,27-hexadeutero-19-nor-cholecalciferol (1), 1,3-Di-O-acetyl-1,25-dihydroxy-20-cyclopropyl-26,27-hexadeutero-19-nor-cholecalciferol (2), 1,25-Dihydroxy-20-cyclopropyl-26,27-hexadeutero-cholecalciferol (3), 1,3-Di-O-acetyl-1,25-dihydroxy-20-cyclopropyl-26,27-hexadeutero-cholecalciferol (4), 1α-Fluoro-25-hydroxy-20-cyclopropyl-26,27-hexadeutero-cholecalciferol (5), 3-O-acetyl-1α-fluoro-25-hydroxy-20-cyclopropyl-cholecalciferol (6), 1,25-Dihydroxy-16-ene-20 cyclopropyl-26,27-hexadeutero-19-nor-cholecalciferol (7), 1,25-Dihydroxy-16-ene-20-cyclopropyl-26,27-hexadeutero-cholecalciferol (8), and 1α-Fluoro-25-hydroxy-16-ene-20-cyclopropyl-26,27-hexadeutero-cholecalciferol (9).

Preferred compounds of the present invention are summarized in Table 1.

TABLE 1

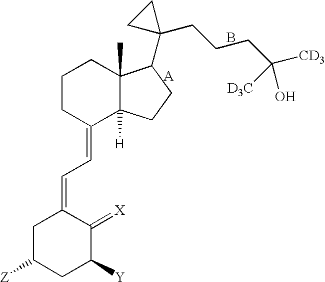

| Compound | X | Z | Y | A | B |
|---|---|---|---|---|---|
| (1) | H₂ | OH | OH | — | — |
| (2) | H₂ | OAc | OAc | — | — |
| (3) | CH₂ | OH | OH | — | — |
| (4) | CH₂ | OAc | OAc | — | — |
| (5) | CH₂ | OH | F | — | — |
| (6) | CH₂ | OAc | F | — | — |
| (7) | H₂ | OH | OH | — | — |
| (8) | CH₂ | OH | OH | — | — |
| (9) | CH₂ | OH | F | — | — |

The structures of some of the compounds of the invention include asymmetric carbon atoms. Accordingly, the isomers arising from such asymmetry (e.g., all enantiomers and diastereomers) are included within the scope of the invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### 3. Uses of the Vitamin D₃ Compounds of the Invention

In one aspect, the invention provides a method for treating a subject for a vitamin D₃ associated state, comprising administering to said subject in need thereof an effective amount of a vitamin D₃ compound of formula I the invention, including compounds of formula I, and the preferred compounds herein above described, such that said subject is treated for said vitamin D₃ associated state.

In one embodiment, the method, further comprises the step of obtaining the vitamin D₃ compound. In another embodiment, the method further comprising identifying a subject in need of treatment for a vitamin D₃ associated state.

In one embodiment, the vitamin D₃ associated state is an ILT3-associated disorder. In a further embodiment, the ILT3-associated disorder is an immune disorder.

In another embodiment, the immune disorder is an autoimmune disorder. In a further embodiment, the autoimmune disorder is selected from the group consisting of type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitis, uveoretinitis, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease.

In another embodiment, the immune disorder is transplant rejection.

In another embodiment, the autoimmune disorder is type I insulin dependent diabetes mellitus.

In yet another embodiment, the vitamin D₃ associated state is a disorder characterized by an aberrant activity of a vitamin D₃-responsive cell. In another embodiment, the disorder comprises an aberrant activity of a hyperproliferative skin cell. In yet another embodiment, the disorder is selected from psoriasis, basal cell carcinoma and keratosis.

In another embodiment, the disorder is psoriasis.

In another embodiment, the disorder comprises an aberrant activity of an endocrine cell. In a further embodiment, the endocrine cell is a parathyroid cell and the aberrant activity is processing and/or secretion of parathyroid hormone.

In yet another embodiment, the disorder is secondary hyperparathyroidism.

In still another embodiment, the disorder comprises an aberrant activity of a bone cell. In a further embodiment, disorder is selected from osteoporosis, osteodystrophy, senile osteoporosis, osteomalacia, rickets, osteitis fibrosa cystica, and renal osteodystrophy. In one embodiment, the disorder is osteoporosis.

In another embodiment, the disorder is cirrhosis or chronic renal disease.

In another embodiment, the disorder is hypertension.

In another embodiment, the invention provides a method wherein the compound of the invention suppresses expression of renin, thereby treating the subject for hypertension. In a further embodiment, the Vitamin D₃ compound is used to suppress rennin expression.

In a further embodiment, compounds used to suppress renin expression include the following:
1,25-Dihydroxy-20-cyclopropyl-26,27-hexadeutero-19-nor-cholecalciferol:

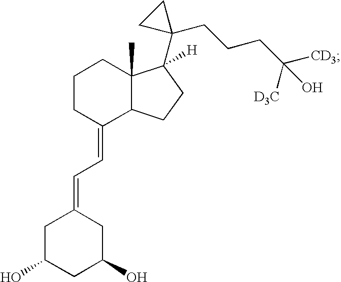

1,3-Di-O-acetyl-1,25-dihydroxy-20-cyclopropyl-26,27-hexadeutero-19-nor-cholecalciferol:

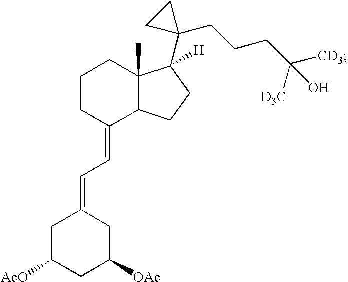

1,25-Dihydroxy-20-cyclopropyl-26,27-hexadeutero-cholecalciferol:

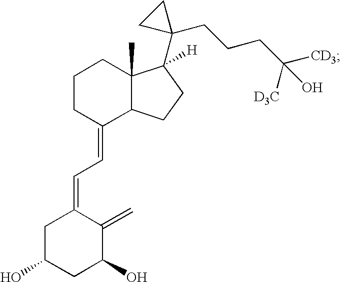

1,3-Di-O-acetyl-1,25-dihydroxy-20-cyclopropyl-26,27-hexadeutero-cholecaciferol:

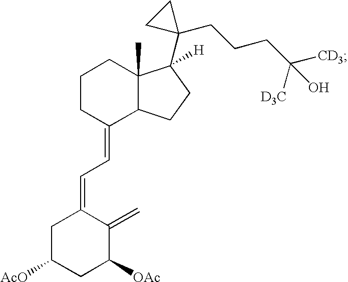

1α-Fluoro-25-hydroxy-20-cyclopropyl-26,27-hexadeutero-cholecalciferol:

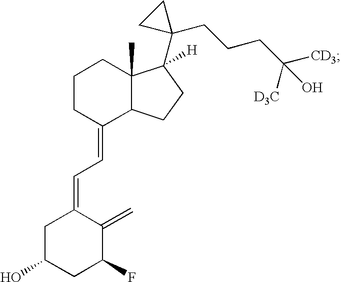

3-O-acetyl-1α-fluoro-25-hydroxy-20-cyclopropyl-cholecalciferol:

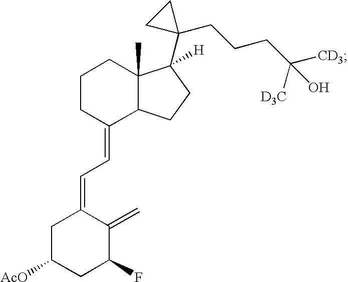

1,25-Dihydroxy-16-ene-20 cyclopropyl-26,27-hexadeutero-19-nor-cholecalciferol:

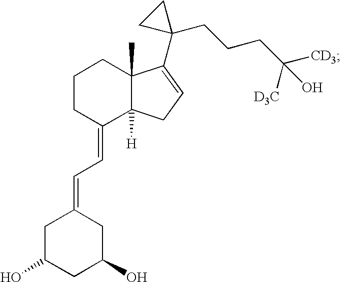

1,25-Dihydroxy-16-ene-20-cyclopropyl-26,27-hexadeutero-cholecalciferol:

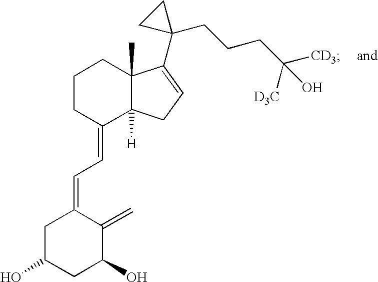

1α-Fluoro-25-hydroxy-16-ene-20-cyclopropyl-26,27-hexadeutero-cholecalciferol:

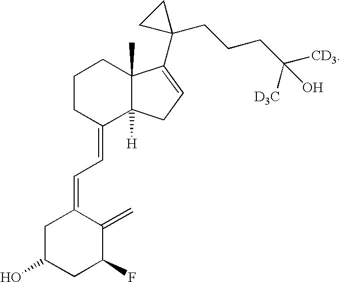

In another embodiment, the disorder is benign prostate hypertrophy.

In another embodiment, the disorder is neoplastic disease. In a further embodiment, the neoplastic disease is selected from the group consisting of leukemia, lymphoma, melanoma, osteosarcoma, colon cancer, rectal cancer, prostate cancer, bladder cancer, and malignant tumors of the lung, breast, gastrointestinal tract, and genitourinary tract. In another embodiment, the neoplastic disease is bladder cancer.

In another embodiment, the disorder is neuronal loss. In a further embodiment, the disorder is selected from the group consisting of Alzheimer's Disease, Pick's Disease, Parkinson's Disease, Vascular Disease, Huntington's Disease, and Age-Associated Memory Impairment.

In another embodiment, the disorder is uveitis.

In another embodiment, the disorder is interstitial cystitis.

In another embodiment, the disorder is characterized by an aberrant activity of a vitamin D₃-responsive smooth muscle cell. In one embodiment, the disorder is uterine myomas. In another embodiment, the disorder is hyperproliferative vascular disease selected from the group consisting of hypertension-induced vascular remodeling, vascular restenosis, and atherosclerosis. In yet another a further embodiment, the disorder is arterial hypertension.

In one embodiment, the invention provides a method of ameliorating a deregulation of calcium and phosphate metabolism, comprising administering to a subject a therapeutically effective amount of a compound of the invention, so as to ameliorate the deregulation of the calcium and phosphate metabolism. In a further embodiment, the deregulation of the calcium and phosphate metabolism leads to osteoporosis.

In yet another embodiment, the invention provides a method of modulating the expression of an immunoglobulin-like transcript 3 (ILT3) surface molecule in a cell, comprising contacting said cell with a compound of the invention in an amount effective to modulate the expression of an immunoglobulin-like transcript 3 (ILT3) surface molecule in said cell. In another embodiment, the cell is within a subject.

In still another embodiment, the invention provides a method of treating an ILT3-associated disorder in a subject, comprising administering to said subject a compound of the invention, in an amount effective to modulate the expression of an ILT3 surface molecule, thereby treating said ILT3-associated disorder in said subject. In one embodiment, the ILT3-associated disorder is an immune disorder. In another embodiment, the immune disorder is an autoimmune disorder. In another embodiment, the autoimmune disorder is type insulin dependent diabetes mellitus.

In one embodiment, the invention provides a method of inducing immunological tolerance in a subject, comprising administering to said subject a compound of the invention, in an amount effective to modulate the expression of an ILT3 surface molecule, thereby inducing immunological tolerance in said subject. In one embodiment, the immunological tolerance is induced in an antigen-presenting cell. In one embodiment, the antigen-presenting cell is selected from the group consisting of dendritic cells, monocytes, and macrophages.

In another embodiment, the invention provides a method of inhibiting transplant rejection in a subject comprising administering to a subject a compound of the invention, in an amount effective to modulate the expression of an ILT3 surface molecule, thereby inhibiting transplant rejection in said subject. In one embodiment, the transplant is a solid organ transplant. In one embodiment, the transplant is a pancreatic islet transplant. In one embodiment, the transplant is a bone marrow transplant.

In another embodiment, the invention provides a method for modulating immunosuppressive activity by an antigen-presenting cell, comprising contacting an antigen-presenting cell with a compound of the invention, in an amount effective to modulate ILT3 surface molecule expression, thereby modulating said immunosuppressive activity by said antigen-presenting cell.

In a further embodiment, the cell is an antigen-presenting cell. In another embodiment, antigen-presenting cell is selected from the group consisting of dendritic cells, monocytes, and macrophages.

In yet another embodiment, the invention provides a method for preventing or treating bladder dysfunction in a subject in need thereof by administering an effective amount of a compound of the invention, thereby to prevent or treat bladder dysfunction in said subject.

In one embodiment, the bladder dysfunction is characterized by the presence of bladder hypertrophy. In another embodiment, the bladder dysfunction is overactive bladder. In another embodiment, the subject is male. In another embodiment, the male is concurrently suffering from BPH. In one embodiment, the subject is female.

In a further embodiment, the invention provides a method wherein the vitamin D₃ compound is administered in combination with a pharmaceutically acceptable carrier.

In yet another embodiment, the invention provides a method wherein said vitamin D₃ compound is administered to the subject using a pharmaceutically-acceptable formulation.

In still another embodiment, the invention provides a method wherein said pharmaceutically-acceptable formulation provides sustained delivery of said vitamin D₃ compound to a subject for at least four weeks after the pharmaceutically-acceptable formulation is administered to the subject.

In one embodiment, the invention provides a method, wherein the expression of said immunoglobulin-like transcript 3 (ILT3) surface molecule is upregulated.

In another embodiment, the invention provides a method wherein the compound is formulated in a pharmaceutical composition together with a pharmaceutically acceptable diluent or carrier. In another embodiment, the invention provides a method, wherein said compound is a Vitamin D receptor agonist.

In another embodiment, the invention provides a method, wherein the subject is a mammal, preferably a human.

In further embodiment, the compound is administered orally. In another embodiment, the compound is administered intravenously. In another embodiment, the compound is administered topically. In another embodiment, the compound is administered parenterally.

In yet another embodiment, the compound is administered at a concentration of 0.001 μg-100 μg/kg of body weight.

In another aspect, the invention provides a pharmaceutical composition, comprising an effective amount of a compound of the invention, and a pharmaceutically acceptable diluent or carrier. In one embodiment, the effective amount is effective to treat a vitamin D₃ associated state. In another embodiment, the invention provides a pharmaceutical composition, wherein said vitamin D₃ associated state is an ILT3-associated disorder. In another embodiment, the invention provides a pharmaceutical composition, wherein said vitamin D₃ associated state is a disorder characterized by an aberrant activity of a vitamin D₃-responsive cell. In another embodiment, the invention provides a pharmaceutical composition, wherein said vitamin D₃ associated state is bladder dysfunction. In another embodiment, the invention provides a pharmaceutical composition, wherein said disorder is hypertension.

In one aspect, the invention provides a packaged formulation for use in the treatment of a vitamin D₃ associated state, comprising a pharmaceutical composition comprising a compound of the invention, and instructions for use in the treatment of a vitamin D₃ associated state. In one embodiment, the invention provides a package formulation wherein said vitamin D₃ associated state is an ILT3-associated disorder. In another embodiment, the invention provides a packaged formulation, wherein said vitamin D₃ associated state is a disorder characterized by an aberrant activity of a vitamin D₃-responsive cell. In another embodiment, the invention provides a packaged formulation, wherein said vitamin D₃ associated state is bladder dysfunction.

In certain embodiments, the methods of the invention include administering to a subject a therapeutically effective amount of a vitamin D₃ compound in combination with another pharmaceutically active compound. Examples of pharmaceutically active compounds include compounds known to treat autoimmune disorders, e.g., immunosuppressant agents such as cyclosporin A, rapamycin, desoxyspergualine, FK 506, steroids, azathioprine, anti-T cell antibodies and monoclonal antibodies to T cell subpopulations. Other pharmaceutically active compounds that may be used can be found in Harrison's Principles of Internal Medicine, Thirteenth Edition, Eds. T. R. Harrison et al. McGraw-Hill N.Y., NY; and the Physicians Desk Reference 50th Edition 1997, Oradell N.J., Medical Economics Co., the complete contents of which are expressly incorporated herein by reference. The vitamin D₃ compound and the pharmaceutically active compound may be administered to the subject in the same pharmaceutical composition or in different pharmaceutical compositions (at the same time or at different times).

A. Hyperproliferative Conditions

In another aspect, the present invention provides a method of treating a subject for a disorder characterized by aberrant activity of a vitamin D₃-responsive cell. The method involves administering to the subject an effective amount of a pharmaceutical composition of a vitamin D₃ compound of formula I or otherwise described herein such that the activity of the cell is modulated.

In certain embodiments, the cells to be treated are hyperproliferative cells. As described in greater detail below, the vitamin D₃ compounds of the invention can be used to inhibit the proliferation of a variety of hyperplastic and neoplastic tissues. In accordance with the present invention, vitamin D₃ compounds of the invention can be used in the treatment of both pathologic and non-pathologic proliferative conditions characterized by unwanted growth of vitamin D₃-responsive cells, e.g., hyperproliferative skin cells, immune cells, and tissue having transformed cells, e.g., such as carcinomas, sarcomas and leukemias. In other embodiments, the cells to be treated are aberrant secretory cells, e.g., parathyroid cells, immune cells.

The use of vitamin D compounds in treating hyperproliferative conditions has been limited because of their hypercalcemic effects. Thus, vitamin D₃ compounds of the invention can provide a less toxic alternative to current methods of treatment.

In one embodiment, the invention features a method for inhibiting the proliferation and/or inducing the differentiation of a hyperproliferative skin cell, e.g., an epidermal or an epithelial cell, e.g., a keratinocytes, by contacting the cells with a vitamin D₃ compound of the invention. In general, the method includes a step of contacting a pathological or non-pathological hyperproliferative cell with an effective amount of such vitamin D₃ compound to promote the differentiation of the hyperproliferative cells. The present method can be performed on cells in culture, e.g., in vitro or ex vivo, or can be performed on cells present in an animal subject, e.g., as part of an in vivo therapeutic protocol. The therapeutic regimen can be carried out on a human or any other animal subject.

The vitamin D₃ compounds of the present invention can be used to treat a hyperproliferative skin disorder. Exemplary disorders include, but are not limited to, psoriasis, basal cell carcinoma, keratinization disorders and keratosis. Additional examples of these disorders include eczema; lupus associated skin lesions; psoriatic arthritis; rheumatoid arthritis that involves hyperproliferation and inflammation of epithelial-related cells lining the joint capsule; dermatitides such as seborrheic dermatitis and solar dermatitis; keratoses such as seborrheic keratosis, senile keratosis, actinic keratosis, photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis.

In an illustrative example, vitamin D₃ compounds of the invention can be used to inhibit the hyperproliferation of keratinocytes in treating diseases such as psoriasis by administering an effective amount of these compounds to a subject in need of treatment. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis.

B. Neoplasia

The invention also features methods for inhibiting the proliferation and/or reversing the transformed phenotype of vitamin D₃-responsive hyperproliferative cells by contacting the cells with a vitamin D₃ compound of formula I or otherwise described herein. In general, the method includes a step of contacting pathological or non-pathological hyperproliferative cells with an effective amount of a vitamin D₃ compound of the invention for promoting the differentiation of the hyperproliferative cells. The present method can be performed on cells in culture, e.g., in vitro or ex vivo, or can be performed on cells present in an animal subject, e.g. as part of an in vivo therapeutic protocol. The therapeutic regimen can be carried out on a human or other subject.

The vitamin D₃ compounds of formula I or otherwise described herein can be tested initially in vitro for their inhibitory effects in the proliferation of neoplastic cells. Examples of cell lines that can be used are transformed cells, e.g., the human promyeloid leukemia cell line HL-60, and the human myeloid leukemia U-937 cell line (Abe E. et al. (1981) Proc. Natl. Acad. Sci. USA 78:4990-4994; Song L. N. and Cheng T. (1992) Biochem Pharmacol 43:2292-2295; Zhou J. Y. et al. (1989) Blood 74:82-93; U.S. Pat. Nos. 5,401,733, U.S. Pat. No. 5,087,619). Alternatively, the antitumoral effects of vitamin D₃ compounds of the invention can be tested in vivo using various animal models known in the art and summarized in Bouillon, R. et al (1995) Endocrine Reviews 16(2):233 (Table E), which is incorporated by reference herein. For example, SL mice are routinely used in the art to test vitamin D compounds as models for MI myeloid leukemia (Honma et al. (1983) Cell Biol. 80:201-204; Kasukabe T. et al. (1987) Cancer Res. 47:567-572); breast cancer studies can be performed in, for example, nude mice models for human MX1 (ER) (Abe J. et al. (1991) Endocrinology 129:832-837; other cancers, e.g., colon cancer, melanoma osteosarcoma, can be characterized in, for example, nude mice models as describe in (Eisman J. A. et al. (1987) Cancer Res. 47:21-25; Kawaura A. et al. (1990) Cancer Lett 55:149-152; Belleli A. (1992) Carcinogenesis 13:2293-2298; Tsuchiya H. et al. (1993) J. Orthopaed Res. 11: 122-130).

The subject method may also be used to inhibit the proliferation of hyperplastic/neoplastic cells of hematopoietic origin, e.g., arising from myeloid, lymphoid or erythroid lineages, or precursor cells thereof. For instance, the present invention contemplates the treatment of various myeloid disorders including, but not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97). Lymphoid malignancies which may be treated by the subject method include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas contemplated by the treatment method of the present invention include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF) and Hodgkin's disease.

In certain embodiments, the vitamin D₃ compounds of the invention can be used in combinatorial therapy with conventional cancer chemotherapeutics. Conventional treatment regimens for leukemia and for other tumors include radiation, drugs, or a combination of both. In addition to radiation, the following drugs, usually in combinations with each other, are often used to treat acute leukemias: vincristine, prednisone, methotrexate, mercaptopurine, cyclophosphamide, and cytarabine. In chronic leukemia, for example, busulfan, melphalan, and chlorambucil can be used in combination. All of the conventional anti-cancer drugs are highly toxic and tend to make patients quite ill while undergoing treatment. Vigorous therapy is based on the premise that unless every leukemic cell is destroyed, the residual cells will multiply and cause a relapse.

The subject method can also be useful in treating malignancies of the various organ systems, such as affecting lung, breast, lymphoid, gastrointestinal, and genito-urinary tract as well as adenocarcinomas which include malignancies such as most colon cancers, renal-cell carcinoma, prostate cancer and/or testicular tumors, non-small cell carcinoma of the lung, cancer of the small intestine, cancer of the esophagus, and bladder cancer.

According to the general paradigm of vitamin D₃ involvement in differentiation of transformed cells, exemplary solid tumors that can be treated according to the method of the present invention include vitamin D₃-responsive phenotypes of sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, bladder cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Determination of a therapeutically effective anti-neoplastic amount or a prophylactically effective anti-neoplastic amount of the vitamin D₃ compound of the invention, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician, the severity of the condition being treated and the particular compound being employed. In determining the therapeutically effective antineoplastic amount or dose, and the prophylactically effective antineoplastic amount or dose, a number of factors are considered by the attending clinician, including, but not limited to: the specific hyperplastic/neoplastic cell involved; pharmacodynamic characteristics of the particular agent and its mode and route of administration; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the vitamin D₃ compounds of the invention with other co-administered therapeutics); and other relevant circumstances. U.S. Pat. No. 5,427,916, for example, describes method for predicting the effectiveness of antineoplastic therapy in individual patients, and illustrates certain methods which can be used in conjunction with the treatment protocols of the instant invention.

Treatment can be initiated with smaller dosages which are less than the optimum dose of the compound. Thereafter, the dosage should be increased by small increments until the optimum effect under the circumstances is reached. For convenience, the total daily dosage may be divided and administered in portions during the day if desired. A therapeutically effective antineoplastic amount and a prophylactically effective anti-neoplastic amount of a vitamin D₃ compound of the invention is expected to vary from about 0.1 milligram per kilogram of body weight per day (mg/kg/day) to about 100 mg/kg/day.

Compounds which are determined to be effective for the prevention or treatment of tumors in animals, e.g., dogs, rodents, may also be useful in treatment of tumors in humans. Those skilled in the art of treating tumors in humans will know, based upon the data obtained in animal studies, the dosage and route of administration of the compound to humans. In general, the dosage and route of administration in humans is expected to be similar to that in animals.

The identification of those patients who are in need of prophylactic treatment for hyperplastic/neoplastic disease states is well within the ability and knowledge of one skilled in the art. Certain of the methods for identification of patients which are at risk of developing neoplastic disease states which can be treated by the subject method are appreciated in the medical arts, such as family history of the development of a particular disease state and the presence of risk factors associated with the development of that disease state in the subject patient. A clinician skilled in the art can readily identify such candidate patients, by the use of, for example, clinical tests, physical examination and medical/family history.

C. Immunological Activity

Healthy individuals protect themselves against foreign invaders using many different mechanisms, including physical barriers, phagocytic cells in the blood and tissues, a class of immune cells known as lymphocytes, and various blood-born molecules. All of these mechanisms participate in defending individuals from a potentially hostile environment. Some of these defense mechanisms, known as natural or innate immunity, are present in an individual prior to exposure to infectious microbes or other foreign macromolecules, are not enhanced by such exposures, and do not discriminate among most foreign substances. Other defense mechanisms, known as acquired or specific immunity, are induced or stimulated by exposure of foreign substances, are exquisitely specific for distinct macromolecules, and increase in magnitude and defensive capabilities with each successive exposure to a particular macromolecule. Substances that induce a specific immune response are known as antigens (see, e.g., Abbas, A. et al., Cellular and Molecular Immunology, W.B. Saunders Company, Philadelphia, 1991; Silverstein, A. M. A history of Immunology, San Diego, Academic Press, 1989; Unanue A. et al., Textbook of Immunology, 2^{nd} ed. Williams and Wilkens, Baltimore, 1984).

One of the most remarkable properties of the immune system is its ability to distinguish between foreign antigens and self-antigens. Therefore, the lymphocytes in each individual are able to recognize and respond to many foreign antigens but are normally unresponsive to the potentially antigenic substances present in the individual. This immunological unresponsiveness is referred to as immune tolerance (see, e.g., Burt R K et al. (2002) Blood 99:768; Coutinho, A. et al. (2001) Immunol. Rev. 182:89; Schwartz, R H (1990) Science 248:1349; Miller, J. F. et al. (1989) Immunology Today 10:53).

Self-tolerance is an acquired process that has to be learned by the lymphocytes of each individual. It occurs in part because lymphocytes pass through a stage in their development when an encounter with antigen presented by antigen-presenting cells (APCs) leads to their death or inactivation in a process known as positive and negative selection (see, e.g., Debatin K M (2001) Ann. Hematol. 80 Suppl 3:B29; Abbas, A. (1991), supra). Thus, potentially self-recognizing lymphocytes come into contact with self-antigens at this stage of functional immaturity and are prevented from developing to a stage at which they would be able to respond to self-antigens. Autoimmunity arises when abnormalities in the induction or maintenance of self-tolerance occur that result in a loss of tolerance to a particular antigen(s) and a subsequent attack by the host's immune system on the host's tissues that express the antigen(s) (see, e.g., Boyton R J et al. (2002) Clin. Exp. Immunol. 127:4; Hagiwara E. (2001) Ryumachi 41:888; Burt R K et al. (2992) Blood 99:768).

The ability of the immune system to distinguish between self and foreign antigens also plays a critical role in tissue transplantation. The success of a transplant depends on preventing the immune system of the host recipient from recognizing the transplant as foreign and, in some cases, preventing the graft from recognizing the host tissues as foreign. For example, when a host receives a bone marrow transplant, the transplanted bone marrow may recognize the new host as foreign, resulting in graft versus host disease (GVHD). Consequently, the survival of the host depends on preventing both the rejection of the donor marrow as well as rejection of the host by the graft immune reaction (see, e.g., Waldmann H et al. (2001) Int. Arch. Allergy Immunol. 126:11).

Currently, deleterious immune reactions that result in autoimmune diseases and transplant rejections are prevented or treated using agents such as steroids, azathioprine, anti-T cell antibodies, and more recently, monoclonal antibodies to T cell subpopulations. Immunosuppressive drugs such as cyclosporin A (CsA), rapamycin, desoxyspergualine and FK-506 are also widely used.

Nonspecific immune suppression agents, such as steroids and antibodies to lymphocytes, put the host at increased risk for opportunistic infection and development of tumors. Moreover, many immunosuppressive drugs result in bone demineralization within the host (see, e.g., Chhajed P N et al. (2002) Indian J. Chest Dis. Allied 44:31; Wijdicks E F (2001) Liver Transpl. 7:937; Karamehic J et al. (2001) Med. Arh. 55:243; U.S. Pat. No. 5,597,563 issued to Beschorner, W E and U.S. Pat. No. 6,071,897 issued to DeLuca H F et al.). Because of the major drawbacks associated with existing immunosuppressive modalities, there is a need for a new approach for treating immune disorders, e.g., for inducing immune tolerance in a host.

Thus, in another aspect, the invention provides a method for modulating the activity of an immune cell by contacting the cell with a vitamin D₃ compound of formula I or otherwise described herein.

In one embodiment, the present invention provides a method for suppressing immune activity in an immune cell by contacting a pathological or non-pathological immune cell with an effective amount of a vitamin D₃ compound of the invention to thereby inhibit an immune response relative to the cell in the absence of the treatment. The present method can be performed on cells in culture, e.g., in vitro or ex vivo, or can be performed on cells present in an animal subject, e.g., as part of an in vivo therapeutic protocol. In vivo treatment can be carried out on a human or other animal subject.

The vitamin D₃ compounds of the invention can be tested initially in vitro for their inhibitory effects on T cell proliferation and secretory activity, as described in Reichel, H. et al., (1987) Proc. Natl. Acad. Sci. USA 84:3385-3389; Lemire, J. M. et al. (1985) J. Immunol 34:2032-2035. Alternatively, the immunosuppressive effects can be tested in vivo using the various animal models known in the art and summarized by Bouillon, R. et al. (1995) Endocrine Reviews 16(2) 232 (Tables 6 and 7). For example, animal models for autoimmune disorders, e.g., lupus, thyroiditis, encephalitis, diabetes and nephritis are described in (Lemire J. M. (1992) J. Cell Biochem. 49:26-31; Koizumi T. et al. (1985) Int. Arch. Allergy Appl. Immunol. 77:396-404; Abe J. et al. (1990) Calcium Regulation and Bone Metabolism 146-151; Fournier C. et al. (1990) Clin. Immunol Immunopathol. 54:53-63; Lemire J. M. and Archer D. C. (1991) J. Clin. Invest. 87:1103-1107); Lemire, J. M. et al., (1994) Endocrinology 135 (6):2818-2821; Inaba M. et al. (1992) Metabolism 41:631-635; Mathieu C. et al. (1992) Diabetes 41:1491-1495; Mathieu C. et al. (1994) Diabetologia 37:552-558; Lillevang S. T. et al. (1992) Clin. Exp. Immunol. 88:301-306, among others). Models for characterizing immunosuppressive activity during organ transplantation, e.g., skin graft, cardiac graft, islet graft, are described in Jordan S. C. et al. (1988) v Herrath D (eds) Molecular, Cellular and Clinical Endocrinology 346-347; Veyron P. et al. (1993) Transplant Immunol 1:72-76; Jordan S. C. (1988) v Herrath D (eds) Molecular, Cellular and Clinical Endocrinology 334-335; Lemire J. M. et al. (1992) Transplantation 54:762-763; Mathieu C. et al. (1994) Transplant Proc. 26:3128-3129).

After identifying certain test compounds as effective suppresors of an immune response in vitro, these compounds can be used in vivo as part of a therapeutic protocol. Accordingly, another aspect of the invention provides a method of suppressing an immune response, comprising administering to a subject a pharmaceutical preparation of a vitamin D₃ compounds of the invention, so as to inhibit immune reactions such as graft rejection, autoimmune disorders and inflammation.

In one embodiment, the invention provides a method for treating a subject for a vitamin D₃ associated state, wherein the vitamin D₃ associated state is an ILT3-associated disorder, by administering to the subject an effective amount of a vitamin D₃ compound of the invention. In one embodiment, the ILT3-associated state is an immune disorder. In certain embodiments, the immune disorder is an autoimmune disorder. In a specific embodiment, the immune disorder is Type 1 diabetes mellitus. In other embodiments, the immune disorder is transplant rejection.

For example, the subject vitamin D₃ compound of the invention can be used to inhibit responses in clinical situations where it is desirable to downmodulate T cell responses. For example, in graft-versus-host disease, cases of transplantation, autoimmune diseases (including, for example, diabetes mellitus, arthritis (including rheumatoid arthritis, juvenile rheumatoid arthritis, osteoarthritis, psoriatic arthritis), multiple sclerosis, encephalomyelitis, diabetes, myasthenia gravis, systemic lupus erythematosis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis). Downmodulation of immune activity will also be desirable in cases of allergy such as, atopic allergy.

Another aspect of the invention provides a method of modulating the expression of an immunoglobulin-like transcript 3 (ILT3) surface molecule in a cell. The method includes contacting the cell with a compound of formula I in an amount effective to modulate the expression of an immunoglobulin-like transcript 3 (ILT3) surface molecule in the cell. In one embodiment, cell is within a subject a subject. In another embodiment the modulation is upregulation of expression. In other embodiment, the modulation is downregulation of expression.

A related aspect of the invention provides a method of treating an ILT3-associated disorder in a subject. The method includes administering to the subject a compound of formula I in an amount effective to modulate the expression of an ILT3 surface molecule, thereby treating the ILT3-associated disorder in the subject.

In certain embodiments, the present invention provides methods and compositions for treating immune disorders, such as, for example, autoimmune disorders and transplant rejections, such as graft versus host disease (GVHD). These embodiments of the invention are based on the discovery that vitamin D₃ compounds of the invention are able to modulate the expression of immunoglobulin-like transcript 3 (ILT3) on cells, e.g., antigen-presenting cells.

Accordingly, another aspect of the invention provides a method for inhibiting transplant rejection in a subject. The method includes administering to the subject a compound of formula I in an amount effective to modulate the expression of an ILT3 surface molecule, thereby inhibiting transplant rejection in the subject. In one embodiment, the transplant is an organ transplant. In another embodiment, the transplant is a pancreatic islet transplant. In yet another embodiment, the transplant is a bone marrow transplant.

As described before, determination of a therapeutically effective immunosuppressive amount can be readily made by the attending clinician, as one skilled in the art, by the use of known techniques and by observing results obtained under analogous circumstances. Compounds which are determined to be effective in animals, e.g., dogs, rodents, may be extrapolated accordingly to humans by those skilled in the art. Starting dose/regimen used in animals can be estimated based on prior studies. For example, doses of vitamin D₃ compounds of the invention to treat autoimmune disorders in rodents can be initially estimated in the range of 0.1 g/kg/day to 1 g/kg/day, administered orally or by injection.

Those skilled in the art will know based upon the data obtained in animal studies, the dosage and route of administration in humans is expected to be similar to that in animals. Exemplary dose ranges to be used in humans are from 0.25 to 10 μg/day, preferably 0.5 to 5 μg/day per adult (U.S. Pat. No. 4,341,774).

D. Calcium and Phosphate Homeostasis

The present invention also relates to a method of treating in a subject a disorder characterized by deregulation of calcium metabolism. This method comprises contacting a pathological or non-pathological vitamin D₃ responsive cell with an effective amount of a vitamin D₃ compound of the invention to thereby directly or indirectly modulate calcium and phosphate homeostasis. Techniques for detecting calcium fluctuation in vivo or in vitro are known in the art.

Exemplary Ca⁺⁺ homeostasis related assays include assays that focus on the intestine where intestinal ⁴⁵Ca²⁺ absorption is determined either 1) in vivo (Hibberd K. A. and Norman A. W. (1969) Biochem. Pharmacol. 18:2347-2355; Hurwitz S. et al. (1967) J. Nutr. 91:319-323; Bickle D. D. et al. (1984) Endocrinology 114:260-267), or 2) in vitro with everted duodenal sacs (Schachter D. et al. (1961) Am. J. Physiol 200:1263-1271), or 3) on the genomic induction of calbindin-D₂₈ₖ in the chick or of calbindin-D₉ₖ in the rat (Thomasset M. et al. (1981) FEBS Lett. 127:13-16; Brehier A. and Thomasset M. (1990) Endocrinology 127:580-587). The bone-oriented assays include: 1) assessment of bone resorption as determined via the release of Ca²⁺ from bone in vivo (in animals fed a zero Ca²⁺ diet) (Hibberd K. A. and Norman A. W. (1969) Biochem. Pharmacol. 18:2347-2355; Hurwitz S. et al. (1967) J. Nutr. 91:319-323), or from bone explants in vitro (Bouillon R. et al. (1992) J. Biol. Chem. 267:3044-3051), 2) measurement of serum osteocalcin levels [osteocalcin is an osteoblast-specific protein that after its synthesis is largely incorporated into the bone matrix, but partially released into the circulation (or tissue culture medium) and thus represents a good market of bone formation or turnover] (Bouillon R. et al. (1992) Clin. Chem. 38:2055-2060), or 3) bone ash content (Norman A. W. and Wong R. G. (1972) J. Nutr. 102:1709-1718). Only one kidney-oriented assay has been employed. In this assay, urinary Ca²⁺ excretion is determined (Hartenbower D. L. et al. (1977) Walter de Gruyter, Berlin pp 587-589); this assay is dependent upon elevations in the serum Ca²⁺ level and may reflect bone Ca²⁺ mobilizing activity more than renal effects. Finally, there is a “soft tissue calcification” assay that can be used to detect the consequences of administration of a compound of the invention. In this assay a rat is administered an intraperitoneal dose of ⁴⁵Ca²⁺, followed by seven daily relative high doses of a compound of the invention; in the event of onset of a severe hypercalcemia, soft tissue calcification can be assessed by determination of the ⁴⁵Ca²⁺ level. In all these assays, vitamin D₃ compounds of the invention are administered to vitamin D-sufficient or -deficient animals, as a single dose or chronically (depending upon the assay protocol), at an appropriate time interval before the end point of the assay is quantified.

In certain embodiments, vitamin D₃ compounds of the invention can be used to modulate bone metabolism. The language “bone metabolism” is intended to include direct or indirect effects in the formation or degeneration of bone structures, e.g., bone formation, bone resorption, etc., which may ultimately affect the concentrations in serum of calcium and phosphate. This term is also intended to include effects of vitamin D₃ compounds in bone cells, e.g. osteoclasts and osteoblasts, that may in turn result in bone formation and degeneration. For example, it is known in the art, that vitamin D₃ compounds exert effects on the bone forming cells, the osteoblasts through genomic and non-genomic pathways (Walters M. R. et al. (1982) J. Biol. Chem. 257:7481-7484; Jurutka P. W. et al. (1993) Biochemistry 32:8184-8192; Mellon W. S. and DeLuca H. F. (1980) J. Biol. Chem. 255:4081-4086). Similarly, vitamin D₃ compounds are known in the art to support different activities of bone resorbing osteoclasts such as the stimulation of differentiation of monocytes and mononuclear phagocytes into osteoclasts (Abe E. et al. (1988) J. Bone Miner Res. 3:635-645; Takahashi N. et al. (1988) Endocrinology 123:1504-1510; Udagawa N. et al. (1990) Proc. Natl. Acad. Sci. USA 87:7260-7264). Accordingly, vitamin D₃ compounds of the invention that modulate the production of bone cells can influence bone formation and degeneration.

The present invention provides a method for modulating bone cell metabolism by contacting a pathological or a non-pathological bone cell with an effective amount of a vitamin D₃ compound of the invention to thereby modulate bone formation and degeneration. The present method can be performed on cells in culture, e.g., in vitro or ex vivo, or can be performed in cells present in an animal subject, e.g., cells in vivo. Exemplary culture systems that can be used include osteoblast cell lines, e.g., ROS 17/2.8 cell line, monocytes, bone marrow culture system (Suda T. et al. (1990) Med. Res. Rev. 7:333-366; Suda T. et al. (1992) J. Cell Biochem. 49:53-58) among others. Selected compounds can be further tested in vivo, for example, animal models of osteopetrosis and in human disease (Shapira F. (1993) Clin. Orthop. 294:34-44).

In a preferred embodiment, a method for treating osteoporosis is provided, comprising administering to a subject a pharmaceutical preparation of a vitamin D₃ compound of the invention to thereby ameliorate the condition relative to an untreated subject.

Vitamin D₃ compounds of the invention can be tested in ovariectomized animals, e.g., dogs, rodents, to assess the changes in bone mass and bone formation rates in both normal and estrogen-deficient animals. Clinical trials can be conducted in humans by attending clinicians to determine therapeutically effective amounts of the vitamin D₃ compounds of the invention in preventing and treating osteoporosis.

In other embodiments, therapeutic applications of the vitamin D₃ compounds of the invention include treatment of other diseases characterized by metabolic calcium and phosphate deficiencies. Exemplary of such diseases are the following: osteoporosis, osteodystrophy, osteomalacia, rickets, osteitis fibrosa cystica, renal osteodystrophy, osteoschlerosis, anti-convulsant treatment, osteopenia, fibrogenesis-imperfecta ossium, secondary hyperparathyroidism, hypoparathyroidism, hyperparathyroidism, cirrhosis, obstructive jaundice, drug induced metabolism, medullary carcinoma, chronic renal disease, hypophosphatemic VDRR, vitamin D-dependent rickets, sarcoidosis, glucocorticoid antagonism, malabsorption syndrome, steatorrhea, tropical sprue, idiopathic hypercalcemia and milk fever.

E. Hormone Secretion

In yet another aspect, the present invention provides a method for modulating hormone secretion of a vitamin D₃-responsive cell, e.g., an endocrine cell. Hormone secretion includes both genomic and non-genomic activities of vitamin D₃ compounds of the invention that control the transcription and processing responsible for secretion of a given hormone e.g., parathyroid hormone (PTH), calcitonin, insulin, prolactin (PRL) and TRH in a vitamin D₃ responsive cell (Bouillon, R. et al. (1995) Endocrine Reviews 16(2):235-237).

The present method can be performed on cells in culture, e.g. in vitro or ex vivo, or on cells present in an animal subject, e.g., in vivo. Vitamin D₃ compounds of the invention can be initially tested in vitro using primary cultures of parathyroid cells. Other systems that can be used include the testing by prolactin secretion in rat pituitary tumor cells, e.g., GH4C1 cell line (Wark J. D. and Tashjian Jr. A. H. (1982) Endocrinology 111:1755-1757; Wark J. D. and Tashjian Jr. A. H. (1983) J. Biol. Chem. 258:2118-2121; Wark J. D. and Gurtler V. (1986) Biochem. J. 233:513-518) and TRH secretion in GH4C1 cells. Alternatively, the effects of vitamin D₃ compounds of the invention can be characterized in vivo using animals models as described in Nko M. et al. (1982) Miner Electrolyte Metab. 5:67-75; Oberg F. et al. (1993) J. Immunol. 150:3487-3495; Bar-Shavit Z. et al. (1986) Endocrinology 118:679-686; Testa U. et al. (1993) J. Immunol. 150:2418-2430; Nakamaki T. et al. (1992) Anticancer Res. 12:1331-1337; Weinberg J. B. and Larrick J. W. (1987) Blood 70:994-1002; Chambaut-Guérin A. M. and Thomopoulos P. (1991) Eur. Cytokine New. 2:355; Yoshida M. et al. (1992) Anticancer Res. 12:1947-1952; Momparler R. L. et al. (1993) Leukemia 7:17-20; Eisman J. A. (1994) Kanis J A (eds) Bone and Mineral Research 2:45-76; Veyron P. et al. (1993) Transplant Immunol. 1:72-76; Gross M. et al. (1986) J Bone Miner Res. 1:457-467; Costa E. M. et al. (1985) Endocrinology 117:2203-2210; Koga M. et al. (1988) Cancer Res. 48:2734-2739; Franceschi R. T. et al. (1994) J. Cell Physiol. 123:401-409; Cross H. S. et al. (1993) Naunyn Schmiedebergs Arch. Pharmacol. 347:105-110; Zhao X. and Feldman D. (1993) Endocrinology 132:1808-1814; Skowronski R. J. et al. (1993) Endocrinology 132:1952-1960; Henry H. L. and Norman A. W. (1975) Biochem. Biophys. Res. Commun. 62:781-788; Wecksler W. R. et al. (1980) Arch. Biochem. Biophys. 201:95-103; Brumbaugh P. F. et al. (1975) Am. J. Physiol. 238:384-388; Oldham S. B. et al. (1979) Endocrinology 104:248-254; Chertow B. S. et al. (1975) J. Clin Invest. 56:668-678; Canterbury J. M. et al. (1978) J. Clin. Invest. 61:1375-1383; Quesad J. M. et al. (1992) J. Clin. Endocrinol. Metab. 75:494-501.

In certain embodiments, the vitamin D₃ compounds of the present invention can be used to inhibit parathyroid hormone (PTH) processing, e.g., transcriptional, translational processing, and/or secretion of a parathyroid cell as part of a therapeutic protocol. Therapeutic methods using these compounds can be readily applied to all diseases, involving direct or indirect effects of PTH activity, e.g., primary or secondary responses.

Accordingly, therapeutic applications for the vitamin D₃ compounds of the invention include treating diseases such as secondary hyperparathyroidism of chronic renal failure (Slatopolsky E. et al. (1990) Kidney Int. 38:S41-S47; Brown A. J. et al. (1989) J. Clin. Invest. 84:728-732). Determination of therapeutically affective amounts and dose regimen can be performed by the skilled artisan using the data described in the art.

F. Protection Against Neuronal Loss

In yet another aspect, the present invention provides a method of protecting against neuronal loss by contacting a vitamin D₃ responsive cell, e.g. a neuronal cell, with a vitamin D₃ compound of the invention to prevent or retard neuron loss. The language “protecting against” is intended to include prevention, retardation, and/or termination of deterioration, impairment, or death of a neurons.

Neuron loss can be the result of any condition of a neuron in which its normal function is compromised. Neuron deterioration can be the result of any condition which compromises neuron function which is likely to lead to neuron loss. Neuron function can be compromised by, for example, altered biochemistry, physiology, or anatomy of a neuron. Deterioration of a neuron may include membrane, dendritic, or synaptic changes which are detrimental to normal neuronal functioning. The cause of the neuron deterioration, impairment, and/or death may be unknown. Alternatively, it may be the result of age- and/or disease-related changes which occur in the nervous system of a subject.

When neuron loss is described herein as “age-related”, it is intended to include neuron loss resulting from known and unknown bodily changes of a subject which are associated with aging. When neuron loss is described herein as “disease-related”, it is intended to include neuron loss resulting from known and unknown bodily changes of a subject which are associated with disease. It should be understood, however, that these terms are not mutually exclusive and that, in fact, many conditions that result in the loss of neurons are both age- and disease-related.

Exemplary age-related diseases associated with neuron loss and changes in neuronal morphology include, for example, Alzheimer's Disease, Pick's Disease, Parkinson's Disease, Vascular Disease, Huntington's Disease, and Age-Associated Memory Impairment. In Alzheimer's Disease patients, neuron loss is most notable in the hippocampus, frontal, parietal, and anterior temporal cortices, amygdala, and the olfactory system. The most prominently affected zones of the hippocampus include the CA1 region, the subiculum, and the entorhinal cortex. Memory loss is considered the earliest and most representative cognitive change because the hippocampus is well known to play a crucial role in memory. Pick's Disease is characterized by severe neuronal degeneration in the neocortex of the frontal and anterior temporal lobes which is sometimes accompanied by death of neurons in the striatum. Parkinson's Disease can be identified by the loss of neurons in the substantia nigra and the locus ceruleus. Huntington's Disease is characterized by degeneration of the intrastriatal and cortical cholinergic neurons and GABA-ergic neurons. Parkinson's and Huntington's Diseases are usually associated with movement disorders, but often show cognitive impairment (memory loss) as well.

Age-Associated Memory Impairment (AAMI) is another age-associated disorder that is characterized by memory loss in healthy, elderly individuals in the later decades of life. Crook, T. et al. (1986) Devel. Neuropsych. 2(4):261-276. Presently, the neural basis for AAMI has not been precisely defined. However, neuron death with aging has been reported to occur in many species in brain regions implicated in memory, including cortex, hippocampus, amygdala, basal ganglia, cholinergic basal forebrain, locus ceruleus, raphe nuclei, and cerebellum. Crook, T. et al. (1986) Devel. Neuropsych. 2(4):261-276.

Vitamin D₃ compounds of the invention can protect against neuron loss by genomic or non-genomic mechanisms. Nuclear vitamin D₃ receptors are well known to exist in the periphery but have also been found in the brain, particularly in the hippocampus and neocortex. Non-genomic mechanisms may also prevent or retard neuron loss by regulating intraneuronal and/or peripheral calcium and phosphate levels. Furthermore, vitamin D₃ compounds of the invention may protect against neuronal loss by acting indirectly, e.g., by modulating serum PTH levels. For example, a positive correlation has been demonstrated between serum PTH levels and cognitive decline in Alzheimer's Disease.

The present method can be performed on cells in culture, e.g. in vitro or ex vivo, or on cells present in an animal subject, e.g., in vivo. Vitamin D₃ compounds of the invention can be initially tested in vitro using neurons from embryonic rodent pups (See e.g. U.S. Pat. No. 5,179,109-fetal rat tissue culture), or other mammalian (See e.g. U.S. Pat. No. 5,089,517-fetal mouse tissue culture) or non-mammalian animal models. These culture systems have been used to characterize the protection of peripheral, as well as, central nervous system neurons in animal or tissue culture models of ischemia, stroke, trauma, nerve crush, Alzheimer's Disease, Pick's Disease, and Parkinson's Disease, among others.

Examples of in vitro systems to study the prevention of destruction of neocortical neurons include using in vitro cultures of fetal mouse neurons and glial cells previously exposed to various glutamate agonists, such as kainate, NMDA, and α-amino-3-hydroxy-5-methyl-4-isoxazolepronate (AMPA). U.S. Pat. No. 5,089,517. See also U.S. Pat. No. 5,170,109 (treatment of rat cortical/hippocampal neuron cultures with glutamate prior to treatment with neuroprotective compound); U.S. Pat. Nos. 5,163,196 and 5,196,421 (neuroprotective excitatory amino acid receptor antagonists inhibit glycine, kainate, AMPA receptor binding in rats).

Alternatively, the effects of vitamin D₃ compounds of the invention can be characterized in vivo using animals models. Neuron deterioration in these model systems is often induced by experimental trauma or intervention (e.g. application of toxins, nerve crush, interruption of oxygen supply).

G. Smooth Muscle Cells

In yet another aspect, the present invention provides a method of modulating the activity of a vascular smooth muscle cell by contacting a vitamin D₃-responsive smooth muscle cell with a vitamin D₃ compound of the invention to activate or, preferably, inhibit the activity of the cell. The language “activity of a smooth muscle cell” is intended to include any activity of a smooth muscle cell, such as proliferation, migration, adhesion and/or metabolism.

In certain embodiments, the vitamin D₃ compounds of the invention can be used to treat diseases and conditions associated with aberrant activity of a vitamin D₃-responsive smooth muscle cell. For example, the present invention can be used in the treatment of hyperproliferative vascular diseases, such as hypertension induced vascular remodeling, vascular restenosis and atherosclerosis. In other embodiments, the compounds of the present invention can be used in treating disorders characterized by aberrant metabolism of a vitamin D₃-responsive smooth muscle cell, e.g., arterial hypertension.

The present method can be performed on cells in culture, e.g. in vitro or ex vivo, or on cells present in an animal subject, e.g., in vivo. Vitamin D₃ compounds of the invention can be initially tested in vitro as described in Catellot et al. (1982), J. Biol. Chem. 257(19): 11256.

### 4. Suppression of Renin Expression

The compounds of the present invention control blood pressure by the suppression of rennin expression and are useful as antihypertensive agents. Renin-angiotensin regulatory cascade plays a significant role in the regulation of blood pressure, electrolyte and volume homeostasis (Y. C. Li, Abstract, DeLuca Symposium on Vitamin D₃, Tauc, N. Mex., Jun. 15-Jun. 19, 2002, p. 18). Thus, the invention provides a method of treating a subject for a vitamin D₃ associated state, wherein the vitamin D₃ associated state is a disorder characterized by an aberrant activity of a cell that expresses renin. The method includes administering to the subject an effective amount of a compound of formula I, such that renin expression by the cell is suppressed, and the subject is thereby treated for hypertension.

### 5. Bladder Dysfunction

Morphological bladder changes, including a progressive de-nervation and hypertrophy of the bladder wall are frequent histological findings in patients with different bladder disorders leading to overactive bladder such as bladder disorders associated with, for example, clinical benign prostatic hyperplasia (BPH) and spinal cord injury.

The increase in tension and/or strain on the bladder observed in these conditions has been shown to be associated with cellular and molecular alterations, e.g., in cytoskeletal and contractile proteins, in mitochondrial function, and in various enzyme activities of the smooth muscle cells. The hypertrophy of the bladder wall also involves alterations in its extracellular matrix and non-smooth muscle components.

These changes in the bladder are associated with the storage (irritative) symptoms, in particular frequency, urgency, urge incontinence and nocturia. These symptoms affect the social, psychological, domestic, occupational, physical and sexual lives of the patients leading to a profound negative impact on their quality of life.

At the present time, an ideal treatment of these symptoms has not been found. Each of the therapeutic options available (for example, anti-muscarinics or alpha-blockers) is associated with disadvantages relating to their mechanism of action, which is based only on the management of symptoms and not on the treatment of the etiology of the condition. In fact, the clinical utility of some of the available agents has been limited by poor efficacy and lack of universal patient acceptance due to a number of significant side effects.

As a consequence there is a need for new treatments that provide improved clinical effectiveness by targeting the underlying etiological factor, the abnormal growth and consequent dysfunction of bladder smooth muscle cells.

As described herein, it has now surprisingly been found that vitamin D analogs can treat and prevent bladder dysfunction in disorders associated with bladder hypertrophy, such as bladder overactivity and clinical BPH. Overactive bladder, also known as detrusor overactivity or detrusor instability, involves involuntary bladder spasms. A hyperactive detrusor muscle can cause overactive bladder. Although the underlying cause of overactive bladder can be neurological disease (e.g. multiple sclerosis, Parkinson's disease, stroke, spinal cord lesions), nerve damage caused by abdominal trauma, pelvic trauma, or surgery, stroke, multiple sclerosis, infection, bladder cancer, drug side effects or enlarged prostate (BPH), in many cases the cause is idiopathic, i.e. of unknown cause.

In addition, such vitamin D related compounds have an application in the treatment of irritative voiding symptoms associated with BPH. BPH is associated not only with enlargement of the gland leading to bladder outlet obstruction (BOO) and symptoms secondary to this, but also to morphological bladder changes, including a hypertrophy of the bladder wall and progressive de-nervation. These changes lead to increased functional demands and disruption of the coordination within the bladder smooth muscle cells.

### 6. Uveitis

Uveitis, a condition comprising inflammation of the eye including the iris, ciliary body, and choroid, actually comprises a large group of diverse diseases affecting not only the uvea but also the retina, optic nerve and vitreous. According to the International Uveitis Study Group, there are several classifications of uveitis: anterior, intermediate, posterior and panuveitis (total). Inflammation may be induced by trauma or toxic or infectious agents, but in most cases the mechanisms seem to be autoimmune in nature. Symptoms may be acute, sub-acute, chronic (greater than 3 months duration) and recurrent. The etiology is unknown in the majority of cases of endogenous uveitis. Uveitis is a major cause of severe visual impairment. Although the number of patients blinded from uveitis is unknown, it has been estimated that uveitis accounts for 10-15% of all cases of total blindness in the USA.

A variety of conditions can be described as posterior uveitis: focal, multifocal or diffuse choroiditis, chorioretinitis, retinochoroiditis, uveoretinitis or neurouveitis. The condition is usually painless but is characterised by the presence of floaters, vision impairment (sudden or gradual) such as blurring of vision, etc., and vision loss. Posterior uveitis may have several etiologies, and manifests itself in complex and sometimes misleading clinical conditions. There is growing evidence both in experimental models and clinically that endogenous posterior uveoretinitis is often characterised by an exaggerated immune response which causes tissue destruction. When no apparent infectious or neoplastic aetiology is found, treatment can be directed towards dampening the resulting inflammatory cascade and hopefully reducing tissue damage. In one embodiment, the invention provides a method of treating uveitis.

### 7. Interstitial Cystitis

Interstitial cystitis, referred to herein as “IC”, is a chronic inflammatory bladder disease, also known as chronic pelvic pain syndrome (CPPS) or painful bladder syndrome (PBS), characterized by pelvic pain, urinary urgency and frequency. This disease affects mainly females, although males are also diagnosed with IC. Unlike other bladder dysfunction conditions, IC is characterized by chronic inflammation of the bladder wall which is responsible for the symptomotology; in other words, the cause of the abnormal bladder contractility and chronic pelvic pain is the chronic inflammation and as a consequence the treatment should target this etiological component. In fact, the traditional treatment of bladder dysfunctions, like overactive bladder, with smooth muscle relaxant agents, is not effective in patients with IC. In one embodiment, the invention provides a method of treating interstitial cystitis.

### 8. Uterine Myomas

Uterine myomas (also known as uterine leiomyomas/leiomyomata, fibroids, myomas/myomata, fibromyomas, myofibromas, fibroleiomyomas) are benign tumours of smooth muscle cells from the uterine myometrium. They include submucous, subserous and intramural myomas. In one embodiment, the invention provides a method for the treatment of uterine myomas.

### 9. Pharmaceutical Compositions

The invention also provides a pharmaceutical composition, comprising an effective amount of a vitamin D₃ compound of formula I or otherwise described herein and a pharmaceutically acceptable carrier. In a further embodiment, the effective amount is effective to treat a vitamin D₃ associated state, as described previously. In an embodiment, the vitamin D₃ compound is administered to the subject using a pharmaceutically-acceptable formulation, e.g., a pharmaceutically-acceptable formulation that provides sustained delivery of the vitamin D₃ compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those vitamin D₃ compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a vitamin D₃ compound(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association a vitamin D₃ compound(s) with the carrier and, optionally, one or more accessory ingredients. In general, the formulations are prepared by uniformly and intimately bringing into association a vitamin D₃ compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a vitamin D₃ compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia, (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the vitamin D₃ compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active vitamin D₃ compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more vitamin D₃ compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a vitamin D₃ compound(s) include powders, sprays, ointments, pastes, creams, lotions, gels, solutions, patches and inhalants. The active vitamin D₃ compound(s) may be mixed under sterile conditions with a pharmaceutically-acceptable carrier, and with any preservatives, buffers, or propellants which may be required.

The ointments, pastes, creams and gels may contain, in addition to vitamin D₃ compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a vitamin D₃ compound(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The vitamin D₃ compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a vitamin D₃ compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the invention.

Pharmaceutical compositions of the invention suitable for parenteral administration comprise one or more vitamin D₃ compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of vitamin D₃ compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the vitamin D₃ compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the vitamin D₃ compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.1 to 10 mg per day.

A preferred dose of the vitamin D₃ compound for the present invention is the maximum that a patient can tolerate and not develop serious hypercalcemia. Preferably, the vitamin D₃ compound of the present invention is administered at a concentration of about 0.001 μg to about 100 μg per kilogram of body weight, about 0.001-about 10 μg/kg or about 0.001 μg-about 100 μg/kg of body weight. Ranges intermediate to the above-recited values are also intended to be part of the invention.

### Exemplification of the Invention

The invention is further illustrated by the following examples which should in no way should be construed as being further limiting.

### Synthesis of Compounds of the Invention

### Experimental

All operations involving vitamin D₃ analogs were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25° C. ¹H NMR spectra were recorded at 400 MHz in CDCl₃ unless indicated otherwise. TLC was carried out on silica gel plates (Merck PF-254) with visualization under short-wavelength UV light or by spraying the plates with 10% phosphomolybdic acid in methanol followed by heating. Flash chromatography was carried out on 40-65 μm mesh silica gel. Preparative HPLC was performed on a 5×50 cm column and 15-30 μM mesh silica gel at a flow rate of 100 ml/min. The results are summarized in Table 1 for compounds 1-9.

### Example 1

### Synthesis of 1,25-Dihydroxy-20-cyclopropyl-26,27-hexadeutero-19-nor-cholecalciferol (1)

### (1R,3aR,4S,7aR)-2-{1-{4-(tert-butyl-dimethyl-silanyloxy)-7a-methyl-octahydro-inden-1-yl}-cyclopropyl}ethyl toluene-4-sulfonic acid ester

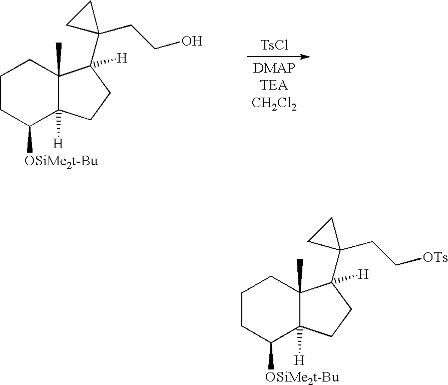

A 100 ml round bottom flask equipped with stir bar and nitrogen sweep was charged with 5.98 g (16.958 mmol) of (1R,3aR,4S,7aR)-2-{1-{4-(tert-Butyl-dimethyl-silanyloxy)-7a-methyl-octahydro-inden-1-yl}-cyclopropyl}-ethanol, 50 ml of dichloromethane, 6 ml of triethylamine and 230 mg (1.883 mmol) of 4-dimethylamino pyridine. A 4.83 g (25.334 mmol) of tosyl chloride was added in one portion. The mixture was stirred at room temperature for 2 h.

The suspension was poured into a mixture of 40 g of ice, 100 ml of saturated sodium hydrogen carbonate solution and 100 ml of hexane. The aqueous layer was re-extracted three times with 50 ml of dichloromethane. These combined extracts were washed with 100 ml of brine, dried over Na₂SO₄ and evaporated.

The residue was purified on a short flash chromatography column using hexane:ethyl acetate (20:1) as mobile phase to give 9.0 g of crude product as colorless oil. Product was used to the next reaction without farther purification.

### (1R,3aR,4S,7aR)-2-(2-{1-{4-(tert-Butyl-dimethyl-silanyloxy)-7a-methyl-octahydro-inden-1-yl}-cyclopropyl}-ethyl)-malonic acid dimethyl ester

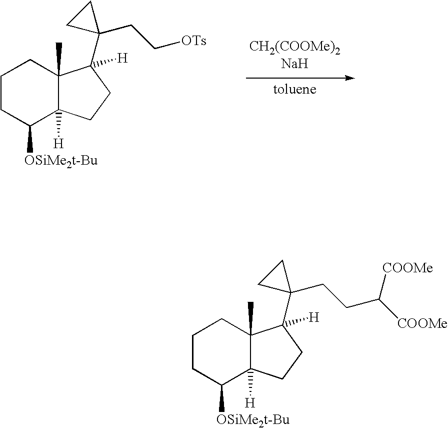

A 500 ml 3-neck round bottom flask equipped with mechanical stirrer, additional funnel with nitrogen sweep and condenser was charged with 160 ml of toluene. A 5.20 g (130 mmol) of sodium hydride (60% dispersion in mineral oil) was added in one portion. To the stirred suspension was added dropwise a solution of 19.36 g (146.5 mmol) of dimethyl malonate in 50 ml of toluene. The gel was heated in 120° C. oil bath for 10 min and then a solution of 9.0 g (ca. 16.958 mmol) of crude (1R,3aR,4S,7aR)-2-{1-{4-(tert-butyl-dimethyl-silanyloxy)-7a-methyl-octahydro-inden-1-yl}-cyclopropyl}ethyl toluene-4-sulfonic acid ester in 100 ml of toluene was added dropwise. The reaction was stirred at this temperature for 6 h.

The flask was placed into an ice bath and 100 ml of cold water was added to dissolve the voluminous precipitate. The mixture was equilibrated with 100 ml of hexane. The resulting aqueous phase was re-extracted three times with 50 ml of toluene. The combined extracts were washed with 100 ml of water and 50 ml of brine, then dried over Na₂SO₄ and evaporated.

The oil residue was chromatographed on column (500 cm³) using hexane:ethyl acetate (20:1; 15:1) as mobile phase and collecting ca. 50 ml fractions. Fractions containing product were pooled and evaporated. The fractions which were mixtures were pooled, evaporated separately and was re-chromatographed on column (300 cm³) using hexane:ethyl acetate (20:1) as mobile phase and collecting ca. 25 ml fractions. Fractions containing product were pooled and evaporated to give 6.148 g (78% for two steps) of product as colorless oil.

### (1R,3aR,4S,7aR)-4-{1-{4-(tert-Butyl-dimethyl-silanyloxy)-7a-methyl-octahydro-inden-1-yl}-cyclopropyl}-butyric acid methyl ester

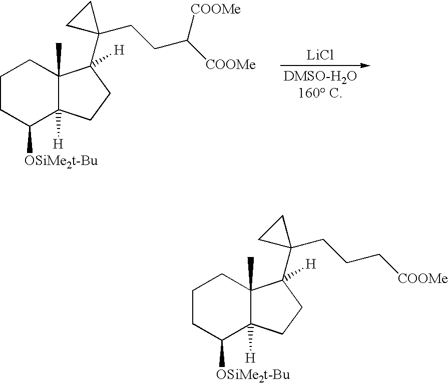

A 100 ml round bottom flask equipped with stir bar and condenser with nitrogen sweep was charged with 6.11 g (13.091 mmol) of (1R,3aR,4S,7aR)-2-(2-{1-{4-(tert-Butyl-dimethyl-silanyloxy)-7a-methyl-octahydro-inden-1-yl}-cyclopropyl}-ethyl)-malonic acid dimethyl ester, 25 ml of mixture of dimethylsulfoxide and water (100:1) and 1.11 g (26.185 mmol) of lithium chloride. The mixture was stirred and heated under nitrogen at 160° C. for 3 h. Then the solution was allowed to cool and distributed between 100 ml of water and 200 ml of hexane The aqueous layer was extracted three times with 50 ml of hexane. The combined organic layers were washed five times with 50 ml of water and 50 ml of brine then dried over Na₂SO₄ and evaporated.

The oil residue was chromatographed on column (500 cm³) using hexane:ethyl acetate (50:1) as mobile phase and collecting ca. 50 ml fractions. Fractions containing product were pooled and evaporated to give of colorless oil. The fractions which were mixtures were pooled, evaporated separately and re-chromatographed on column (160 cm³) using hexane:ethyl acetate (50:1). It gave 4.19 g (78%) of product.

¹H NMR (CDCl₃): 3.98 (1H, br s), 3.66 (3H, s), 2.29-2.23 (2H, m), 2.10-1.75 (5H, m), 1.68-1.22 (10H, m), 0.94 (3H, s), 0.88 (9H, s), 0.71-0.65 (1H, m), 0.61-0.50 (1H, m), 0.21-0.14 (2H, m), 0.00 (3H, s), -0.02 (3H, s), -0.05-0.12 (1H, m).

### (1R,3aR,4S,7aR)-5-{1-{4-(tert-Butyl-dimethyl-silanyloxy)-7a-methyl-octahydro-inden-1-yl}-cyclopropyl}-1,1,1-trideutero-2-trideuteromethyl-pentan-2-ol

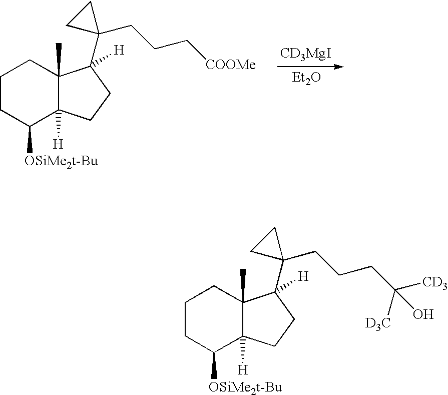

A 250 ml round bottom flask equipped with stir bar, Claisen adapter with rubber septum was charged with 3.012 g (7.370 mmol) of (1R,3aR,4S,7aR)-4-{1-{4-(tert-Butyl-dimethyl-silanyloxy)-7a-methyl-octahydro-inden-1-yl}-cyclopropyl}-butyric acid methyl ester and 75 ml of anhydrous diethyl ether. The solution was cooled in ace-water bath and 20 ml (20 mmol) of 1M methyl-d₃-magnesium iodide in diethyl ether was added dropwise. After completion of the addition the mixture was stirred at room temperature for 1.5 h then cooled again in an ice bath. A 25 ml of saturated solution of ammonium chloride was added dropwise. The resulting precipitate was dissolved by the addition of 100 ml of water. The aqueous layer was re-extracted three times with 50 ml of diethyl ether. The combined ether layers were dried over Na₂SO₄ and evaporated.

The oil residue was chromatographed on column (350 cm³) using hexane:ethyl acetate (9:1) as mobile phase and collecting ca. 50 ml fractions. Fractions containing product were pooled and evaporated to give of colorless oil. The fractions which were mixtures were pooled, evaporated separately and re-chromatographed on column (100 cm³) using hexane:ethyl acetate (9:1). It gave 2.95 g (96%) of product.

¹H NMR (CDCl₃): 3.99 (1H, br s), 2.05-1.76 (4H, m), 1.68-1.17 (14H, m), 0.95 (3H, s), 0.88 (9H, s), 0.70-0.52 (2H, m), 0.22-0.12 (2H, m), 0.01 (3H, s), -0.01 (3H, s), -0.05-0.11 (1H, m).

### (1R,3aR,4S,7aR)-1-{1-(4-Hydroxy-5,5,5-tridutero-4-trideuteromethyl-pentyl)-cyclopropyl}-7a-methyl-octahydro-inden-4-ol

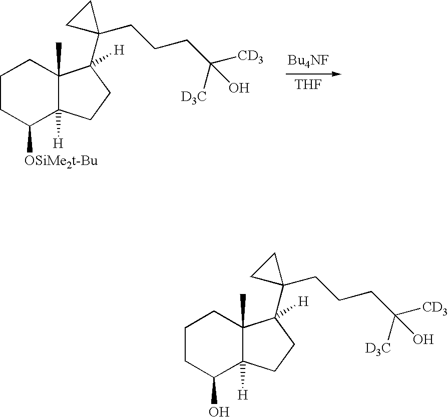

A 50 ml round bottom flask equipped with stir bar and Claisen adapter with rubber septum was charged with 2.940 g, (7.088 mmol) of (1R,3aR,4S,7aR)-5-{1-{4-(tert-Butyl-dimethyl-silanyloxy)-7a-methyl-octahydro-inden-1-yl}-cyclopropyl}-1,1,1-trideutero-2-trideuteromethyl-pentan-2-ol and 25 ml (25.0 mmol) of 1.0M tetrabutylammonium fluoride in tetrahydrofuran. The reaction mixture was stirred at 70° C. for 22 h and the new portion 10 ml (10.0 mmol) of tetrabutylammonium fluoride was added. The reaction was stirred at 70° C. for next 26 h.

The mixture was dissolved by the addition of 150 ml of ethyl acetate and washed six times with 40 ml of water and 20 ml of brine, dried over Na₂SO₄ and evaporated.

The oil residue was chromatographed on column (250 cm³) using hexane:ethyl acetate (3:1) as mobile phase. Fractions containing product were pooled and evaporated to give 2.00 g (94%) of product.

### (1R,3aR,7aR)-1-{1-(4-Hydroxy-5,5,5-trideutero-4-trideuteromethyl-pentyl)-cyclopropyl}-7a-methyl-octahydro-inden-4-one

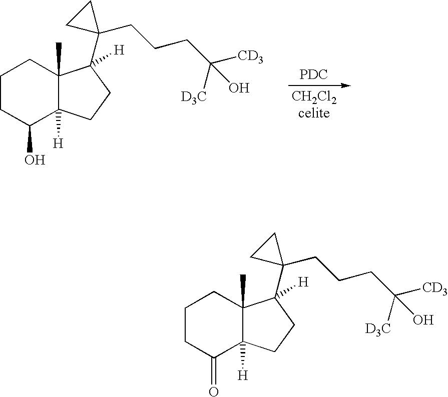

A 250 ml round bottom flask equipped with stir bar and Claisen adapter with rubber septum was charged with 7.42 g (19.723 mmol) of pyridinium dichromate, 7.28 g of celite and 75 ml of dichloromethane. A 1.96 g (6.522 mmol) of (1R,3aR,4S,7aR)-1-[1-(4-Hydroxy-5,5,5-tridutero-4-trideuteromethyl-pentyl)-cyclopropyl]-7a-methyl-octahydro-inden-4-ol in 5 ml of dichloromethane was added dropwise and mixture was stirred in room temperature for 6 h.

The reaction mixture was filtrated through column with 100 cm³ of silica gel using dichloromethane and dichloromethane:ethyl acetate (4:1, 3:1, 2:1) as mobile phases.

The fractions containing product were pooled and evaporated to give 1.92 g (98%) of ketone.

¹H NMR (CDCl₃): 2.50 (1H, dd, J=11.4, 7.0 Hz), 2.29-2.12 (4H, m), 2.05-1.86 (3H, m), 1.75-1.17 (9H, m), 1.08-0.98 (1H, m), 0.73-0.60 (2H, m), 0.69 (3H, s), 0.26-0.19 (2H, m), 0.06-0.01 (1H, m).

### (1R,3aR,7aR)-7a-Methyl-1-{1-(5,5,5-trideutero-4-trideuteromethyl-4-trimethylsilanyloxy-pentyl)-cyclopropyl}-octahydro-inden-4-one

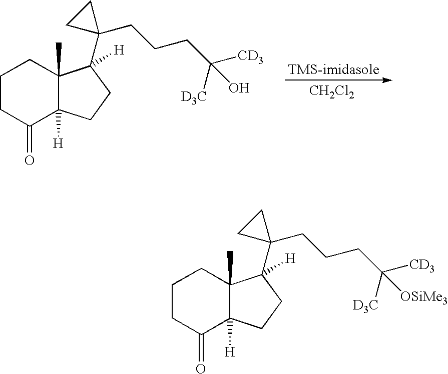

A 100 ml round bottom flask equipped with stir bar and Claisen adapter with rubber septum was charged with 1.91 g (6.399 mmol) of (1R,3aR,7aR)-1-{1-(4-Hydroxy-5,5,5-trideutero-4-trideuteromethyl-pentyl)-cyclopropyl}-7a-methyl-octahydro-inden-4-one and 60 ml of dichloromethane. A 3.8 ml (25.90 mmol) of 1-(trimethylsilyl)imidasole was added dropwise. The mixture was stirred at room temperature for 1 h 45 min.

A 25 ml of water was added and the mixture was stirred for 10 min. The resulting mixture was dissolved by the addition of 200 ml of water. The aqueous layer was extracted five times with 50 ml of ethyl acetate. The combined organic layers were washed with 50 ml of brine, dried over Na₂SO₄ and evaporated.

The oil residue was chromatographed on column (200 cm³) using hexane:dichloromethane (2:1, 1:1) and dichloromethane as mobile phases. Fractions containing product were pooled and evaporated to give 2.10 g (89%) of product as colorless oil.

### 1α,25-Dihydroxy-20-cyclopropyl-26,27-hexadeutero-19-nor-cholecalciferol (1)

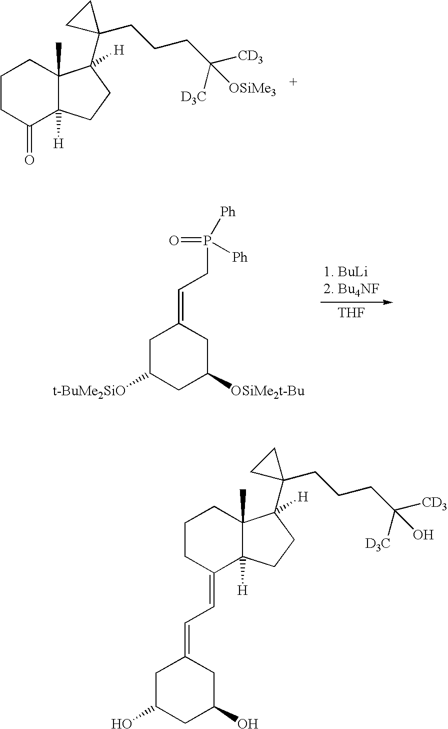

A 50 ml round bottom flask equipped with stir bar and Claisen adapter with rubber septum was charged with 2.155 g (3.776 mmol) of (1R,3R)-1,3-bis-(tert-butyldimethyl)silanyloxy)-5-[2-(diphenylfosphinoyl)ethylidene]-cyclohexane and 15 ml of anhydrous tetrahydrofurane. The reaction mixture was cooled to −78° C. and 2.3 ml (3.68 mmol) 1.6M n-butyllithium in hexane was added dropwise. The resulting deep red solution was stirred at −78° C. for 20 min and 700 mg (1.888 mmol) of (1R,3aR,7aR)-7a-Methyl-1-{1-(5,5,5-trideutero-4-trideuteromethyl-4-trimethylsilanyloxy-pentyl)-cyclopropyl}-octahydro-inden-4-one was added dropwise in 2 ml of anhydrous tetrahydrofurane. The reaction mixture was stirred for 4 h and then the bath was removed and the mixture was poured into 50 ml of ethyl acetate and 50 ml brine. The water fraction was extracted three times with 75 ml of ethyl acetate. All organic layers were combined, dried over Na₂SO₄ and evaporated. The oil residue was chromatographed on column (100 cm³, protected from light) using hexane:ethyl acetate (20:1) as mobile phase. Fractions containing product were pooled and evaporated to give colorless oil which was treated with 20 ml 1.0M tetrabutylammonium fluoride in tetrahydrofurane. The reaction mixture was stirred at room temperature for 24 h.

The mixture was dissolved by the addition of 150 ml of ethyl acetate. The organic layer was washed five times with 50 ml of water and 50 ml of brine, dried over Na₂SO₄ and evaporated.

The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 525 mg (66%) of white foam.

[α]³⁰_{D}=+47.8 c 0.46, CHCl₃

UV λmax (EtOH): 243 nm (ε 32133), 251 nm (ε 37757), 261 nm (ε 25993)

¹H NMR (CDCl₃): 6.30 (1H, d, J=11.3 Hz), 5.82 (1H, d, J=11.3), 4.15-4.08 (1H, m), 4.07-4.00 (1H, m), 2.82-2.78 (1H, m), 2.73 (1H, dd, J=13.1, 3.7 Hz), 2.48 (1H, dd, J=13.3, 3.3 Hz), 2.24-1.24 (21H, m), 1.19 (1H, s), 1.00-0.91 (1, m), 0.68-0.61 (2H, m), 0.59 (3H, s), 0.23-0.17 (2H, m), 0.05-0.05 (1H, m)

| | | | |
|---|---|---|---|
| MS HRES | Calculated for: C₂₇H₃₈D₆FO₃ | [M + Na]⁺ | 445.3559 |
| | Observed: | [M + Na]⁺ | 445.3561 |

### Example 2

### Synthesis of Acetic acid 1α-acetoxy-25-hydroxy-20-cyclopropyl-26,27-hexadeutero-19-nor-cholecalciferyl ester (2)

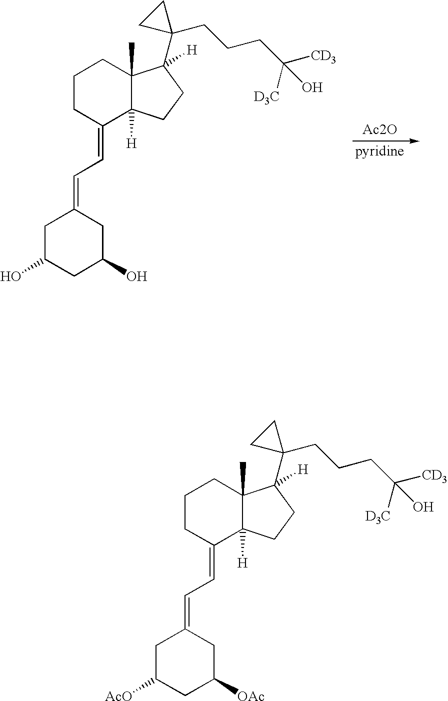

A 25 ml round bottom flask equipped with stir bar and Claisen adapter with rubber septum was charged with 245 mg (0.579 mmol) of 11,25-Dihydroxy-20-cyclopropyl-26,27-hexadeutero-19-nor-cholecalciferol and 6 ml of pyridine. The mixture was stirred at 0-5° C. and 1 ml (10.6 mmol) of acetic anhydride was added dropwise. The reaction mixture was stirred at 0-5° C. for 17 h and new portion 0.75 ml (7.9 mmol) of acetic anhydride was added dropwise. The reaction mixture was stirred for next 24 h.

The mixture was dissolved by the addition of 10 ml of water, stirred for 15 min and poured into 100 ml of ethyl acetate. The mixture was extracted five times with 50 ml of water and 50 ml of brine, dried over Na₂SO₄ and evaporated.

The oil residue was chromatographed on column (50 cm³, protected from light) using hexane:ethyl acetate (2:1) as mobile phase. Fractions containing product were pooled and evaporated to give product as colorless oil. The product was dissolved in methyl acetate and evaporated (4 times) to give 259 mg (88%) of white foam.

[α]³⁰_{D}=+8.2 c 0.45, CHCl₃

UV λmax (EtOH): 243 nm (ε 34931), 251 nm (ε 40870), 260 nm (ε27807)

¹ NMR(CDCl₃): 6.25 (1H, d, J=11.1 Hz), 5.72 (1H, d, J=11.5 Hz), 5.12-5.06 (2H, m), 2.80-2.76 (1H, m), 2.60-2.44 (3H, m), 2.27 (1H, dd, J=13.5, 7.7 Hz), 2.14-1.87 (6H, m), 2.03 (3H, s), 2.00 (3H, s), 1.70-1.25 (11H, m), 1.18 (1H, s), 1.00-0.91 (1H, m), 0.68-0.60 (2H, m), 0.57 (3H, s), 0.23-0.16 (2H, m), 0.00-0.06 (1H, m)

| | | | |
|---|---|---|---|
| MS HRES | Calculated for: C₃₁H₄₂D₆FO₅ | [M + Na]⁺ | 529.3770 |
| | Observed: | [M + Na]⁺ | 529.3782 |

### Example 3

### 1α,25-Dihydroxy-20-cyclopropyl-26,27-hexadeutero-cholecalciferol (3)

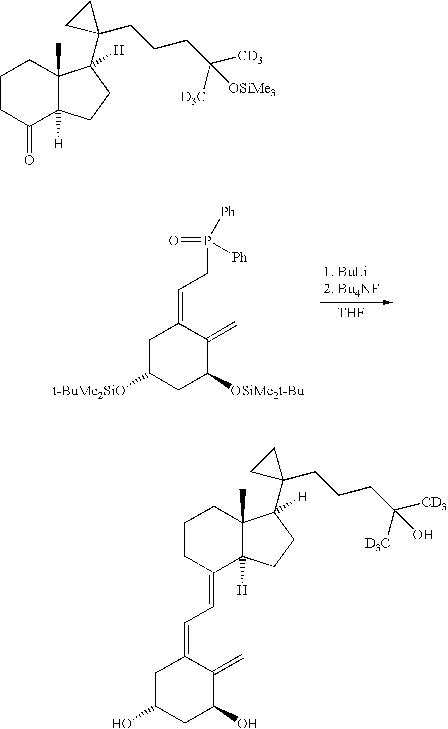

A 50 ml round bottom flask equipped with stir bar and Claisen adapter with rubber septum was charged with 2.201 g (3.774 mmol) of (1S,5R)-1,5-bis-((tert-butyldimethyl)silanyloxy)-3-[2-(diphenylfosphinoyl)-eth-(Z)-ylidene]-2-methylene-cyclohexane and 15 ml of anhydrous tetrahydrofurane. The reaction mixture was cooled to −78° C. and 2.3 ml (3.68 mmol) 1.6M n-butyllithium in hexane was added dropwise. The resulting deep red solution was stirred at −78° C. for 20 min and 700 mg (1.888 mmol) of (1R,3aR,7aR)-7a-Methyl-1-{1-(5,5,5-trideutero-4-trideuteromethyl-4-trimethylsilanyloxy-pentyl)-cyclopropyl}-octahydro-inden-4-one was added dropwise in 2 ml of anhydrous tetrahydrofurane. The reaction mixture was stirred for 4 h and then the bath was removed and the mixture was poured into 60 ml of ethyl acetate and 50 ml of brine. The water fraction was extracted four times with 75 ml of ethyl acetate, dried over Na₂SO₄ and evaporated. The oil residue was chromatographed on column (100 cm³, protected from light) using hexane:ethyl acetate (20:1) as mobile phase. Fractions containing product were pooled and evaporated to give colorless oil which was treated with 20 ml 1.0M tetrabutylammonium fluoride in tetrahydrofurane. The reaction mixture was stirred at room temperature for 24 h. The mixture was dissolved by the addition of 150 ml of ethyl acetate and washed five times with 50 ml of water and 50 ml of brine, dried over Na₂SO₄ and evaporated. The oil residue was chromatographed on column (75 cm³, protected from light) using ethyl acetate as mobile phase. Fractions containing product were pooled and evaporated to give product as colorless oil. Some fractions contain impurity were purified on column (50 cm³, protected from light) using ethyl acetate:hexane (2:1) as mobile phase. The product was dissolved in methyl acetate and evaporated (4 times) to give 749 mg (91%) of white foam.

[α]³⁰_{D}=+3.3 c 0.46, CHCl₃

UV λmax (EtOH): 213 nm (ε 12528), 264 nm (ε 14832)

¹H NMR (CDCl₃): 6.37 (1H, d, J=11.5 Hz), 5.99 (1H, d, J=1.1), 5.32 (1H, s), 4.99 (1H, s), 4.44-4.42 (1H, m), 4.23 (1H, br s), 2.84-2.80 (1H, m), 2.59 (1H, dd, J=13.5, 3.5 Hz), 2.31 (1H, dd, J=13.4, 6.4 Hz), 2.13-2.09 (1H, m), 2.06-1.88 (5H, m), 1.73-1.26 (13H, m), 1.18 (1H, br s), 0.99-0.90 (1H, m), 0.68-0.61 (2H, m), 0.59 (3H, s), 0.21-0.16 (2H, m), 0.00-0.06 (1H, m)

| | | | |
|---|---|---|---|
| MS HRES | Calculated for: C₂₈H₃₈D₆FO₃ | [M + Na]⁺ | 457.3559 |
| | Observed: | [M + Na]⁺ | 457.3563 |

### Example 4

### Acetic acid 1α-acetoxy-25-hydroxy-20-cyclopropyl-26,27-hexadeutero-cholecalciferyl ester (4)

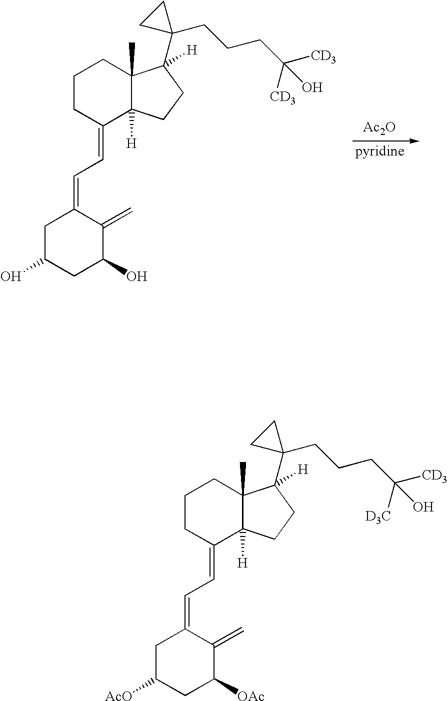

A 25 ml round bottom flask equipped with stir bar and Claisen adapter with rubber septum was charged with 345 mg (0.794 mmol) of 1α25-Dihydroxy-20-cyclopropyl-26,27-hexadeutero-cholecalciferol and 7 ml of pyridine. The mixture was stirred at 0-5° C. and 1.5 ml (15.9 mmol) of acetic anhydride was added dropwise. The reaction mixture was stirred at 0-5° C. for 17 h and new portion 0.5 ml (5.3 mmol) of acetic anhydride was added. The next portion 1 ml (10.6 mmol) of acetic anhydride was added after next 25 h. The reaction mixture was stirred for additional 16 h.

The mixture was dissolved by the addition of 15 ml of water, stirred for 15 min and poured into 120 ml of ethyl acetate. The mixture was extracted five times with 50 ml of water and 50 ml of brine, dried over Na₂SO₄ and evaporated.

The oil residue was chromatographed on column (75 cm³, protected from light) using hexane:ethyl acetate (2:1) as mobile phase. Fractions containing product were pooled and evaporated to give product as colorless oil. The product was dissolved in methyl acetate and evaporated (4 times) to give 364 mg (88%) of white foam.

[α]³⁰_{D}=−20.2 c 0.46, CHCl₃

UV mλax (EtOH): 207 nm (ε 14863), 250 nm (ε 15225), 265 nm (ε 15985)

¹H NMR (CDCl₃): 6.34 (1H, d, J=11.3 Hz), 5.89 (1H, d, J=11.5 Hz), 5.47 (1H, dd, J=6.2, 4.0 Hz), 5.30 (1H, s), 5.21-5.15 (1H, m), 5.03 (1H, d, J=1.7 Hz), 2.82-2.78 (1H, m), 2.64 (1H, dd, J=13.2, 4.3 Hz), 2.38-2.33 (1H, m), 2.13-1.92 (6H, m), 2.05 (3H, s), 2.03 (3H, s), 1.72-1.28 (11H, m), 1.19 (1H, s), 0.98-0.88 (1H, m), 0.68-0.59 (2H, m), 0.56 (3H, s), 0.22-0.16 (2H, m), 0.01-0.06 (1H, m)

| | | | |
|---|---|---|---|
| MS HRES | Calculated for: C₃₂H₄₂D₆FO₅ | [M + Na]⁺ | 541.3770 |
| | Observed: | [M + Na]⁺ | 541.3764 |

### Example 5

### 1α-Fluoro-25-hydroxy-20-cyclopropyl-26,27-hexadeutero-cholecalciferol (5)

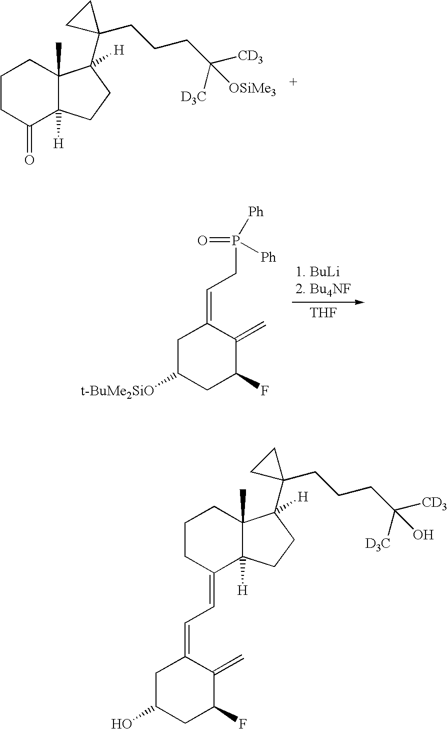

A 50 ml round bottom flask equipped with stir bar and Claisen adapter with rubber septum was charged with 1.907 g (4.052 mmol) of (1S,5R)-1-((tert-butyldimethyl)silanyloxy)-3-[2-(diphenylfosphinoyl)-eth-(Z)-ylidene]-5-fluoro-2-methylene-cyclohexane and 15 ml of anhydrous tetrahydrofurane. The reaction mixture was cooled to −78° C. and 2.5 ml (4.00 mmol) 1.6M n-butyllithium in hexane was added dropwise. The resulting deep red solution was stirred at −78° C. for 20 min and 650 mg (1.754 mmol) of (1R,3aR,7aR)-7a-Methyl-1-{1-(5,5,5-trideutero-4-trideuteromethyl-4-trimethylsilanyloxy-pentyl)-cyclopropyl}-octahydro-inden-4-one was added dropwise in 2 ml of anhydrous tetrahydrofurane. The reaction mixture was stirred for 3.5 h and then the bath was removed and the mixture was poured into 60 ml of ethyl acetate and 50 ml of brine. The water fraction was extracted four times with 60 ml of ethyl acetate dried (Na₂SO₄) and evaporated. The oil residue was chromatographed on column (75 cm^{,} protected from light) using hexane:ethyl acetate-20:1 as mobile phase. Fractions containing product were pooled and evaporated to give colorless oil which was treated with 20 ml 1.0M tetrabutylammonium fluoride in tetrahydrofurane. The reaction mixture was stirred at room temperature for 7.5 h. The mixture was dissolved by the addition of 150 ml of ethyl acetate and washed five times with 50 ml of water and 50 ml of brine, dried over Na₂SO₄ and evaporated. The oil residue was chromatographed on column (75 cm³, protected from light) using hexane:ethyl acetate (1:1) as mobile phase. Fractions containing product were pooled and evaporated to give product as colorless oil. Some fractions contain impurity were purified on column (50 cm³, protected from light) using hexane:ethyl acetate (2:1) as mobile phase. The product was dissolved in methyl acetate and evaporated (4 times) to give 629 mg (82%) of white foam.

[α]³⁰_{D}=+22.1 c 0.43, CHCl₃

UV λmax (EtOH): 209 nm (ε 14376), 243 nm (ε 13949), 269 nm (ε 14083)

¹H NMR (CDCl₃): 6.39 (1H, d, J=11.1 Hz), 6.00 (1H, d, J=11.1 Hz), 5.38 (1H, s), 5.13 (1H, ddd, J=49.5, 6.9, 3.7 Hz), 5.09 (1H, s), 4.22 (1H, br s), 2.84-2.80 (1H, m), 2.62 (1H, dd, J=13.3, 3.7 Hz), 2.30 (1H, dd, J=13.3, 7.5 Hz), 2.23-1.92 (6H, m), 1.74-1.26 (12H, m), 1.18 (1H, s), 0.98-0.91 (1H, m), 0.68-0.61 (2H, m), 0.59 (3H, s), 0.21-0.16 (2H, m), 0.00-0.06 (1H, m)

| | | | |
|---|---|---|---|
| MS HRES | Calculated for: C₂₈H₃₇D₆FO₂ | [M + Na]⁺ | 459.3516 |
| | Observed: | [M + Na]⁺ | 459.3521 |

### Example 6

### Acetic acid 1α-fluoro-25-hydroxy-20-cyclopropyl-26,27-hexadeutero-cholecalciferyl ester (6)

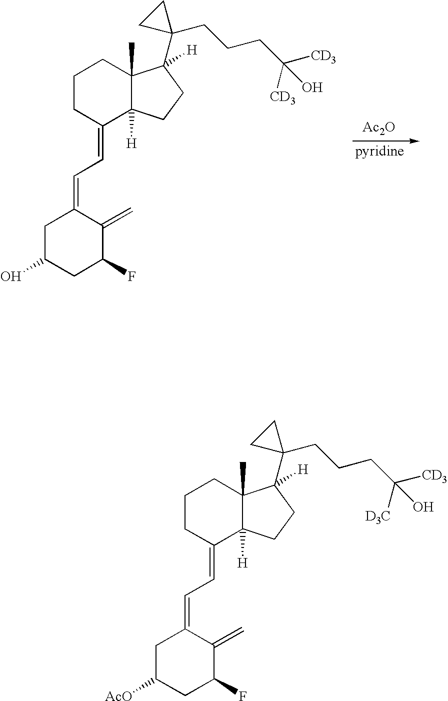

A 25 ml round bottom flask equipped with stir bar and Claisen adapter with rubber septum was charged with 300 mg (0.687 mmol) of 1α-Fluoro-25-hydroxy-20-cyclopropyl-26,27-hexadeutero-cholecalciferol and 6 ml of pyridine. The mixture was stirred at 0-5° C. and 1 ml (10.6 mmol) of acetic anhydride was added dropwise. The reaction mixture was stirred at 0-5° C. for 16 h and new portion 0.5 ml (5.3 mmol) of acetic anhydride was added. The reaction mixture was stirred for next 3 h.

The mixture was dissolved by the addition of 15 ml of water, stirred for 15 min and poured into 120 ml of ethyl acetate. The mixture was extracted five times with 50 ml of water and 50 ml of brine, dried over Na₂SO₄ and evaporated.

The oil residue was chromatographed on column (50 cm³, protected from light) using hexane:ethyl acetate (3:1) as mobile phase. Fractions containing product were pooled and evaporated to give product as colorless oil. The product was dissolved in methyl acetate and evaporated (4 times) to give 292 mg (89%) of white foam.

[α]³⁰_{D}=−15.9 c 0.46, CHCl₃

UV λmax (EtOH): 210 nm (ε 11176), 245 nm (ε 10496), 264 nm (ε 10387)

¹H NMR (CDCl₃): 6.36 (1H, d, J=11.3 Hz), 6.00 (1H, d, J=11.3 Hz), 5.40 (1H, s), 5.23-5.16 (1H, m), 5.10 (1H, dm, J=49.7 Hz), 5.10 (1H, s), 2.82-2.79 (1H, m), 2.64 (1H, dd, J=13.7, 3.7 Hz), 2.41-2.36 (1H, m), 2.23-1.93 (6H, m), 2.04 (3H, s), 1.73-1.26 (12H, m), 0.99-0.92 (1H, m), 0.68-0.61 (2H, m), 0.60 (3H, s), 0.22-0.17 (2H, m), 0.00-0.06 (1H, m)

| | | | |
|---|---|---|---|
| MS HRES | Calculated for: C₃₀H₃₉D₆FO₃ | [M + Na]⁺ | 501.3621 |
| | Observed: | [M + Na]⁺ | 501.3619 |

### Example 7

### Synthesis of 1,25-Dihydroxy-16-ene-20 cyclopropyl-26,27-hexadeutero-19-nor-cholecalciferol (7)

### (3aR,4S,7aR)-1-E/Z-{1-{4-(tert-Butyl-dimethyl-silanyloxy)-7a-methyl-3a,4,5,6,7,7a-hexahydro-3H-inden-1-yl})-cyclopropyl}-2-methoxy-vinyl

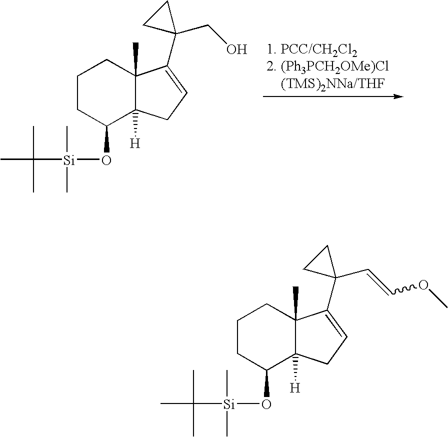

To a stirred suspension of pyridinium chlorochromate (10.3 g, 47.7 mmol) in dichloromethane (100 mL) at room temperature was added dropwise a solution of (3aR,4S,7aR)-{1-{4-(tert-Butyl-dimethyl-silanyloxy)-7a-methyl-3a,4,5,6,7,7a-hexahydro-3H-inden-1-yl}-cyclopropyl}-methanol (6.5 g, 19.31 mmol) in dichloromethene (10.0 mL). The reaction mixture was stirred for 1.0 h and filtered through Celite/Silca gel column (20 g+50 g), which was then washed with 10% AcOEt in hexane to give crude (3aR,4S,7aR)-1-{4-(tert-Butyl-dimethyl-silanyloxy)-7a-methyl-3a,4,5,6,7,7a-hexahydro-3H-inden-1-yl}-cyclopropanecarbaldehyde (5.6 g).

To a stirred suspension of (methoxymethyl)triphenylphosphonium chloride (7.5 g, 21.88 mmol) in tetrahydrofurane (150 mL) at 0° C. was added dropwise sodium bis(trimethylsilyl)amide (22 mL, 22 mmol, 1.0 M in THF). After 30 min. at 0° C. the solution of (3aR,4S,7aR)-1-{4-(tert-Butyl-dimethyl-silanyloxy)-7a-methyl-3a,4,5,6,7,7a-hexahydro-3H-inden-1-yl}-cyclopropanecarbaldehyde (5.6 g, 16.74 mmol) in tetrahydrofurane (20 mL) was added dropwise. The reaction mixture was stirred for 1 h at 0° C., then water (150 mL) was added and the reaction was extracted with hexane (2×150 mL) and dried over Na₂SO₄. The residue (12.5 g) after evaporation of the solvent was purified by FC (200 g, hexane, 5% AcOEt in hexane) to give the titled compound (5.41 g, 14.92 mmol, 77%)

### (3aR,4S,7aR)-1-{1-{4-(tert-Butyl-dimethyl-silanyloxy)-7a-methyl-3a,4,5,6,7,7a-hexahydro-3H-inden-1-yl})-cyclopropyl}-ethynyl

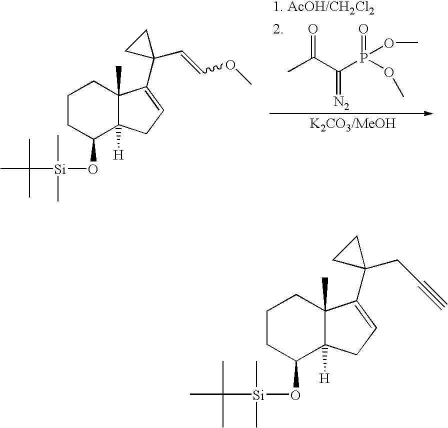

To a stirred solution of (3aR,4S,7aR)-1-E/Z-{1-{4-(tert-Butyl-dimethyl-silanyloxy)-7a-methyl-3a,4,5,6,7,7a-hexahydro-3H-inden-1-yl})-cyclopropyl}-2-methoxy-vinyl (5.41 g, 14.92 mmol) in dichloromethene (50 mL) at room temperature was added acidic acid (25 mL) and the reaction mixture was heated at reflux for 72 hours. NaHCO_{3aq}(350 mL) was added and the reaction mixture was extracted with dichloromethane (2×200 mL), washed with brine (200 mL) and dried over Na₂SO₄) The residue after evaporation of the solvent (1.2 g) was purified by FC (150 g, hexane, 2% AcOEt in hexane) to give (3aR,4S,7aR)-{1-{4-(tert-Butyl-dimethyl-silanyloxy)-7a-methyl-3a,4,5,6,7,7a-hexahydro-3H-inden-1-yl}-cyclopropyl}-acetaldehyde (3.65 g, 10.47 mmol).

To a stirred solution of (3aR,4S,7aR)-{1-{4-(tert-Butyl-dimethyl-silanyloxy)-7a-methyl-3a,4,5,6,7,7a-hexahydro-3H-inden-1-yl}-cyclopropyl}-acetaldehyde (3.65 g, 10.47 mmol) in methanol (15 mL) at room temperature was added (1-diazo-2-oxo-propyl)-phosphonic acid dimethyl ester (3.0 g, 15.61 mmol) in methanol (5 mL). The resulting mixture was cooled in an ice bath and potassium carbonate (3.07 g, 22.21 mmol, powdered) was added. The reaction mixture was stirred in the ice bath for 30 min and then at room temperature for 45 min. Water was added (100 mL) and the mixture was extracted with hexane (2×150 mL). The combined extracts were washed with brine (100 mL) and dried over Na₂SO₄. The residue after evaporation of the solvent (3.9 g) was purified by FC (100 g, hexane, 2% AcOEt in hexane) to give the titled compound (2.6 g, 7.54 mmol, 72%)

[α]²⁸_{D}=+29.8 c 0.8, CHCl₃

¹H NMR (CDCl₃): 5.45 (1H, br. s), 4.04 (1H, br. s), 2.40 (2H, m), 2.24 (1H, m), 1.96-1.38 (9H, m), 1.17 (3H, s), 0.88 (9H, s), 0.74-0.54 (4H, m), 0.01 (6H, s);

¹³C NMR (CDCl₃): 156.44 (0),125.39 (1), 82.65 (1), 69.39 (0), 69.23 (1), 55.92 (1), 47.60 (0), 36.42 (2), 34.65 (2), 30.76 (2), 26.04 (2), 20.34 (3), 19.35 (0), 18.30 (2), 11.516 (2), 10.97 (2), −4.55 (3), −4.87 (3);

| | | | |
|---|---|---|---|
| MS HREI | Calculated for C₂₂H₃₆OSi | M+ | 344.2535 |
| | Observed | M+ | 344.2539 |

### (3aR,4S,7aR)-7a-Methyl-1-{1-(5,5,5-trideutero-4-hydroxy-4-trideuteromethyl-pent-2-ynyl)-cyclopropyl}-3a,4,5,6,7,7a-hexahydro-3H-inden-4-ol

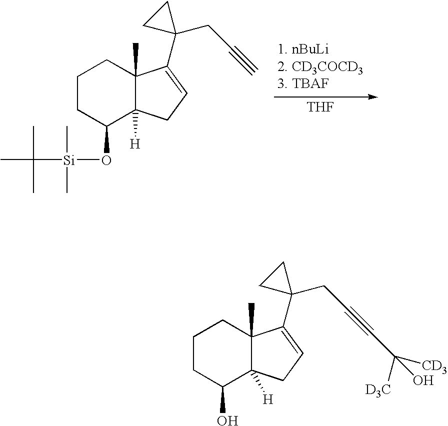

To a stirred solution of (3aR,4S,7aR)-1-{1-{4-(tert-Butyl-dimethyl-silanyloxy)-7a-methyl-3a,4,5,6,7,7a-hexahydro-3H-inden-1-yl})-cyclopropyl}-ethynyl (1.6 g, 4.64 mmol) in tetrahydrofurane (22 mL) at −78° C. was added n-BuLi (4.35 mL, 6.96 mmol, 1.6M in hexane). After stirring at −78° C. for 1 h., acetone-d₆ (1.0 mL, 13.6 mmol, (D, 99, 96) was added and the stirring was continued for 2.5 h. NH₄Cl_{aq} was added (15 mL) and the mixture was stirred for 15 min at room temperature then extracted with AcOEt (2×50 mL). The combined extracts were washed with brine (50 mL) and dried over Na₂SO₄. The residue after evaporation of the solvent (2.4 g) was purified by FC (50 g, 10% AcOEt in hexane) to give (3aR,4S,7aR)-5-{1-{4-(tert-Butyl-dimethyl-silanyloxy)-7a-methyl-3a,4,5,6,7,7a-hexahydro-3H-inden-1-yl}-cyclopropyl}-1,1,1-trideutero-2-trideuteromethyl-pent-3-yn-2-ol (1.81 g, 4.43 mmol) which was treated with tetrabutylammonium fluoride (12 mL, 12 mmol, 1.0M in THF) and stirred at 65-75° C. for 48 h. The mixture was diluted with AcOEt (25 mL) and washed with water (5×25 mL), brine (25 mL). The combined aqueous washes were extracted with AcOEt (25 mL) and the combined organic extracts were dried over Na₂SO₄)

The residue after evaporation of the solvent (2.5 g) was purified by FC (100 g, 20% AcOEt in hexane) to give the titled compound (1.21 g, 4.11 mmol, 89%)

[α]³⁰_{D}=+2.0 c 0.35, CHCl₃

¹H NMR (CDCl₃): 5.47 (1H, m), 4.15 (1H, m), 2.40 (2H, s), 2.28 (1H, ddd, J=13.4, 11.9, 1.5 Hz), 1.98-1.36 (10H, m), 1.19 (3H, s), 0.70-0.52 (4H, m);

¹³C NMR (CDCl₃): 156.32 (0), 125.22 (1), 86.36 (0), 80.33 (0), 69.31 (1), 69.14 (0), 55.20 (1), 47.01 (0), 35.87 (2), 33.70 (2), 29.99 (2), 27.34 (2), 19.39 (2), 19.29 (0), 17.83 (3), 11.05 (2), 10.50 (2);

| | | | |
|---|---|---|---|
| MS HREI | Calculated for C₁₉H₂₂O₂D₆ | M+ | 294.2466 |
| | Observed | M+ | 294.2474 |

### (3aR,4S,7aR)-7a-Methyl-1-{1-(5,5,5-trideutero-4-hydroxy-4-trideuteromethyl-pent-2Z-enyl)-cyclopropyl}-3a,4,5,6,7,7a-hexahydro-3H-inden-4-ol

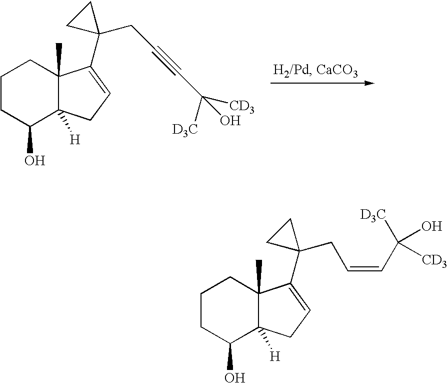

The mixture of (3aR,4S,7aR)-7a-Methyl-1-{1-(5,5,5-trideutero-4-hydroxy-4-trideuteromethyl-pent-2-ynyl)-cyclopropyl}-3a,4,5,6,7,7a-hexahydro-3H-inden-4-ol (1.02 g, 3.46 mmol), ethyl acetate (14 mL), hexane (31 mL), absolute ethanol (1.25 mL), quinoline (66 μL) and Lindlar catalyst (222 mg, 5% Pd on CaCO₃) was hydrogenated at room temperature for 2 h. The reaction mixture was filtered through a celite pad and the pad was washed with AcOEt. The filtrates and the washes were combined and washed with 1M HCl, NaHCO₃ and brine. After drying over Na₂SO₄ the solvent was evaporated and the residue (1.2 g) was purified by FC (75 g, 20% AcOEt in hexane) to give the titled compound (890 mg, 3.0 mmol, 87%)

[α]²⁸_{D}=+1.7 c 0.48, CHCl₃

¹H NMR (CDCl₃): 5.45 (1H, dt, J=11.9, 1.8 Hz), 5.42 (1H, m,), 5.36 (1H, dt, J=12.1, 6.3 Hz), 4.14 (1H, m), 2.43 (1H, m), 2.27 (1H, ddd, J=13.6, 12.2, 1.7 Hz), 2.00-1.24 (11H, m), 1.18 (3H, s), 0.70-0.36 (4H, m);

¹³C NMR (CDCl₃): 156.67 (0), 136.58 (1), 128.65 (1), 125.21 (1), 71.48 (0), 69.37 (1), 55.28 (1), 47.07 (0), 35.89 (2), 35.57 (2), 33.68 (2), 30.04 (2), 21.14 (0), 19.37 (3), 17.84 (2), 11.85 (2),11.06 (2);

| | | | |
|---|---|---|---|
| MS HRES | Calculated for C₁₉H₂₄O₂D₆ | M + H | 296.2622 |
| | Observed | M + H | 296.2619 |

### (3aR,4S,7aR)-7a-Methyl-1-{1-(5,5,5-trideutero-4-hydroxy-4-trideuteromethyl-pentyl)-cyclopropyl}-3a,4,5,6,7,7a-hexahydro-3H-inden-4-ol

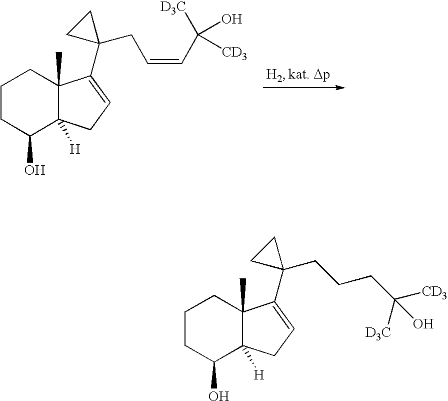

The mixture of (3aR,4S,7aR)-7a-Methyl-1-{1-(5,5,5-trideutero-4-hydroxy-4-trideuteromethyl-pent-2Z-enyl)-cyclopropyl}-3a,4,5,6,7,7a-hexahydro-3H-inden-4-ol (860 mg, 2.9 mmol), 1,4-bis(diphenyl-phosphino)butane 1,5 cyclooctadiene rhodium tetrafluoroborate (200 mg, 0.28 mmol), dichloromethane (35 mL) and one drop of mercury was hydrogenated using Paar apparatus at room temperature and 50 p.s.i. pressure for 2 h. The reaction mixture was filtered through Celite pad, which was then washed with ethyl acetate. The combine filtrates and washes were evaporated to dryness (950 mg) and purified three times by FC (100 g, 20% AcOEt in hexane) to give the titled compound (600 mg, 2.01 mmol, 69%)

[α]³⁰_{D}=−5.3 c 0.45, CHCl₃

¹H NMR (CDCl₃): 5.37 (1H, m,), 4.14 (1H, m), 2.32-1.20 (17H, m), 1.18 (3H, s), 0.64-0.26 (4H, m);

¹³C NMR (CDCl₃): 156.84 (0), 124.87 (1), 70.79 (0), 69.39 (1), 55.42 (1), 47.19 (0), 43.75 (2), 38.31 (2), 35.86 (2), 33.69 (2), 29.97 (2), 22.35 (2), 21.14 (0), 19.46 (3), 17.88 (2), 12.19 (2), 11.28 (2);

| | | | |
|---|---|---|---|
| MS HREI | Calculated for C₁₉H₂₆O₂D₆ | M + H | 298.2779 |
| | Observed | M + H | 298.2787 |

### (3aR,7aR)-7a-Methyl-1-{1-(5,5,5-trideutero-4-trideuteromethyl-4-trimethylsilanyloxy-pentyl)-cyclopropyl}-3a,4,5,6,7,7a-hexahydro-3H-inden-4-one

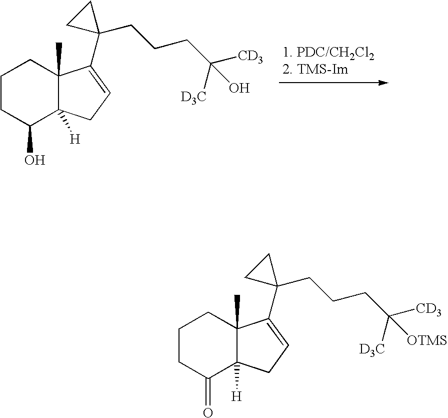

To a stirred suspension of (3aR,4S,7aR)-7a-Methyl-1-{1-(5,5,5-trideutero-4-hydroxy-4-trideuteromethyl-pentenyl)-cyclopropyl}-3a,4,5,6,7,7a-hexahydro-3H-inden-4-ol (450 mg, 1.51 mmol) and Celite (2.0 g) in dichloromethane (10 mL) at room temperature wad added pyridinium dichromate (1.13 g, 3.0 mmol). The resulting mixture was stirred for 3.5 h filtered through silica gel (10 g), and then silica gel pad was washed with 25% AcOEt in hexane. The combined filtrate and washes were evaporated, to give a crude (3aR,7aR)-7a-Methyl-1-{1-(5,5,5-trideutero-4-hydroxy-4-trideuteromethyl-pentenyl)-cyclopropyl}-3a,4,5,6,7,7a-hexahydro-3H-inden-4-one (425 mg, 1.44 mmol, 95%). To a stirred solution of (3aR,7aR)-7a-Methyl-1-{1-(5,5,5-trideutero-4-hydroxy-4-trideuteromethyl-pentenyl)-cyclopropyl}-3a,4,5,6,7,7a-hexahydro-3H-inden-4-one (425 mg, 1.44 mmol) in dichloromethane (10 mL) at room temperature was added trimethylsilyl-imidazole (0.44 mL, 3.0 mmol). The resulting mixture was stirred for 1.0 h filtered through silica gel (15 g) and the silica gel pad was washed with 10% AcOEt in hexane. Combined filtered and washes were evaporated to give the titled compound (450 mg, 1.22 mmol, 85%)

[α]²⁹_{D}=−14.2 c 0.43, CHCl₃

¹H NMR (CDCl₃): 5.33 (1H, dd, J=3.2, 1.5 Hz), 2.81 (1H, dd, J=10.7, 6.4 Hz), 2.44 (1H, ddd, J=15.8, 10.7, 1.6 Hz), 2.30-1.12 (13H, m) overlapping 2.03 (ddd, J=15.9, 6.4, 3.2 Hz), 0.92 (3H, s), 0.66-0.28 (4H, m), 0.08 (9H, s);

¹³C NMR (CDCl₃): 210.74 (0), 155.41 (0), 124.81 (1), 73.71 (0), 64.37 (1), 53.92 (0), 44.67 (2), 40.46 (2), 38.21 (2), 34.80 (2), 26.86 (2), 24.06 (2), 22.28 (2), 21.28 (0), 18.40 (3), 12.59 (2), 10.69 (2), 2.62 (3);

| | | | |
|---|---|---|---|
| MS HRES | Calculated for C₂₂H₃₂O₂SiD₆ | M+ | 368.0318 |
| | Observed | M+ | 368.3029 |

### 1α,25-Dihydroxy-16-ene-20-cyclopropyl-26,27-hexadeutero-19-nor-cholecalciferol (7)

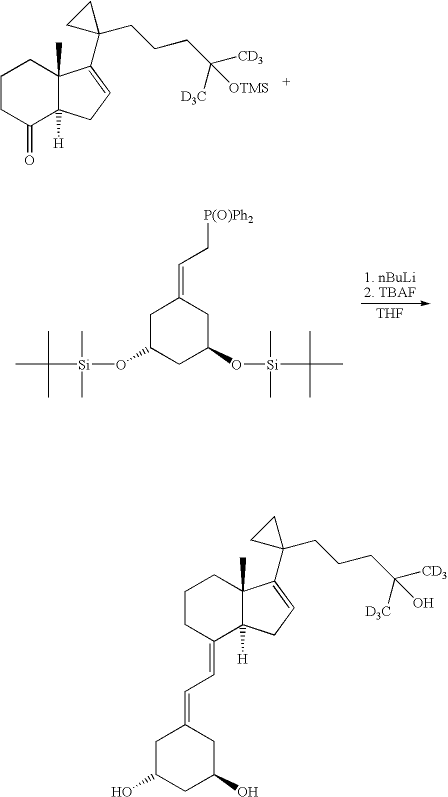

To a stirred solution of a (1R,3R)-1,3-bis-((tert-butyldimethyl)silanyloxy)-5-[2-(diphenylphosphinoyl)ethylidene]-cyclohexane (536 mg, 0.92 mmol) in tetrahydrofurane (7 mL) at −78° C. was added n-BuLi (0.58 mL, 0.93 mmol). The resulting mixture was stirred for 15 min and solution of (3aR,7aR)-7a-Methyl-1-{1-(5,5,5-trideutero-4-trideuteromethyl-4-trimethylsilanyloxy-pentyl)-cyclopropyl}-3a,4,5,6,7,7a-hexahydro-3H-inden-4-one (170 mg, 0.46 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (725 mg) after evaporation of the solvent was purified by FC (15 g, 5% AcOEt in hexane) to give 1α,3β-Di(tert-Butyl-dimethyl-silanyloxy)-25-trimethylsilanyloxy-16-ene-20-cyclopropyl-26,27-hexadeutero-19-nor-cholecalciferol (293 mg, 041 mmol). To the 1α,3β-Di(tert-Butyl-dimethyl-silanyloxy)-25-trimethylsilanyloxy-16-ene-20-cyclopropyl-26,27-hexadeutero-19-nor-cholecalciferol (293 mg, 0.41 mmol) tetrabutylammonium fluoride (4 mL, 4 mmol, 1M solution in THF) was added, at room temperature. The mixture was stirred for 40 h. diluted with AcOEt (25 mL) and washed with water (5×20 mL), brine (20 mL) and dried over Na₂SO₄. The residue (280 mg) after evaporation of the solvent was purified by FC (15 g, 50% AcOEt in hexane and AcOEt) to give the titled compound (163 mg, 0.39 mmol, 84%)

[α]²⁹_{D}=+65.8 c 0.40, EtOH

UV λmax (EtOH): 243 nm (ε 32702251 nm (ε 39060), 261 nm (ε 26595);

¹H NMR (CDCl₃): 6.30 (1H, d, J=11.3 Hz), 5.93 (1H, d, J=11.3 Hz), 5.36 (1H, m), 4.13 (1H, m), 4.05 (1H, m), 2.76 (2H, m), 2.52-1.10 (22H, m), 0.79 (3H, s),0.66-0.24 (4H, m);

¹³C NMR (CDCl₃): 157.05 (0), 142.25 (0), 131.15 (0), 124.66 (1), 123.70 (1), 115.44 (1), 70.82 (0), 67.42 (1), 67.22 (1), 59.51 (1), 50.17 (0), 44.66 (2), 43.79 (2), 42.22 (2), 38.18 (2), 37.25 (2), 35.64 (2), 29.19 (2), 28.56 (2), 23.55 (2), 22.31 (2), 21.37 (0), 18.04 (3), 12.81 (2), 10.38 (2);

| | | | |
|---|---|---|---|
| MS HRES | Calculated for C₂₇H₃₆O₃D₆ | M+ | 420.3511 |
| | Observed | M+ | 420.3524 |

### Example 8

### 1α,25-Dihydroxy-16-ene-20-cyclopropyl-26,27-hexadeutero-cholecalciferol (8)

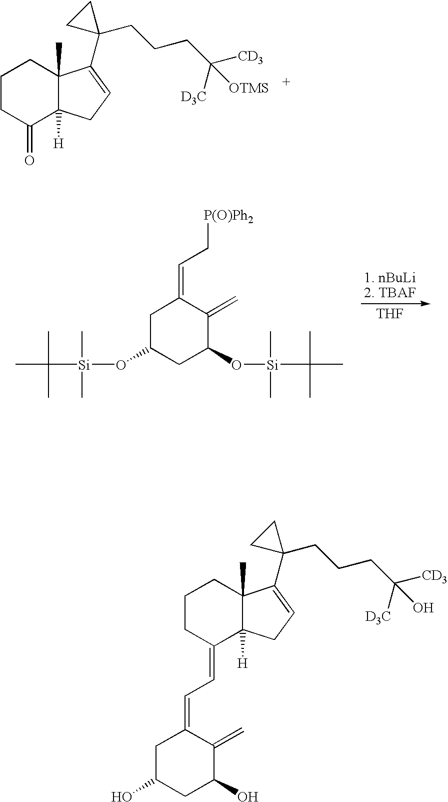

To a stirred solution of a (1S,5R)-1,5-bis-((tert-butyldimethyl)silanyloxy)-3-[2-(diphenylphosphinoyl)-eth-(Z)-ylidene]-2-methylene-cyclohexane (536 mg, 0.92 mmol) in tetrahydrofurane (7 mL) at −78° C. was added n-BuLi (0.58 mL, 0.93 mmol). The resulting mixture was stirred for 15 min and solution of (3aR,7aR)-7a-Methyl-1-[1-(5,5,5-trideutero-4-trideuteromethyl-4-trimethylsilanyloxy-pentyl)-cyclopropyl]-3a,4,5,6,7,7a-hexahydro-3H-inden-4-one (170 mg, 0.46 mmol, in tetrahydrofurane (2 mL) was added dropwise. The reaction mixture was stirred at −72° C. for 3.5 h diluted with hexane (35 mL) washed brine (30 mL) and dried over Na₂SO₄. The residue (725 mg) after evaporation of the solvent was purified by FC (15 g, 5% AcOEt in hexane) to give 1α,3β-Di(tert-Butyl-dimethyl-silanyloxy)-25-trimethylsilanyloxy-16-ene-20-cyclopropyl-26,27-hexadeutero-cholecalciferol (302 mg, 041 mmol). To the 1α,3β-Di(tert-Butyl-dimethyl-silanyloxy)-25-trimethylsilanyloxy-16-ene-20-cyclopropyl-26,27-hexadeutero-cholecalciferol (302 mg, 0.41 mmol) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (280 mg) after evaporation of the solvent was purified by FC (15 g, 50% AcOEt in hexane and AcOEt) to give the titled compound (160 mg, 0.37 mmol, 80%)

[α]²⁹_{D}=+15.3 c 0.34, EtOH

UV λmax (EtOH): 207 nm (ε 17011), 264 nm (ε 15067);

¹H NMR (CDCl₃): 6.37 (1H, d, J=11.3 Hz), 6.09 (1H, d, J=11.3 Hz), 5.33 (2H, m), 5.01 (1H, s), 4.44 (1H, m), 4.23 (1H, m), 2.80 (1H, dd, J=11.9, 3.2 Hz), 2.60 (1H, dd, J=13.2, 3.2 Hz), 2.38-1.08 (20H, m), 0.79 (3H, s),0.66-0.24 (4H, m);

¹³C NMR (CDCl₃): 156.97 (0), 147.53 (0), 142.41 (0), 132.94 (0), 124.83 (1), 124.68 (1), 117.14 (1), 111.60 (2), 70.82 (0), 70.71 (1), 66.88 (1), 59.55 (1), 50.30 (0), 45.23 (2), 43.79 (2),42.90 (2), 38.18 (2), 35.64 (2), 29.19 (2), 28.71 (2), 23.63 (2), 22.30 (2), 21.36 (0), 17.91 (3), 12.82 (2), 10.39 (2);

| | | | |
|---|---|---|---|
| MS HRES | Calculated for C₂₈H₃₆O₃D₆ | M+ | 432.3510 |
| | Observed | M+ | 432.3517 |

### Example 9

### 1α-fluoro-25-hydroxy-16-ene-20-cyclopropyl-26,27-hexadeutero-cholecalciferol (9)

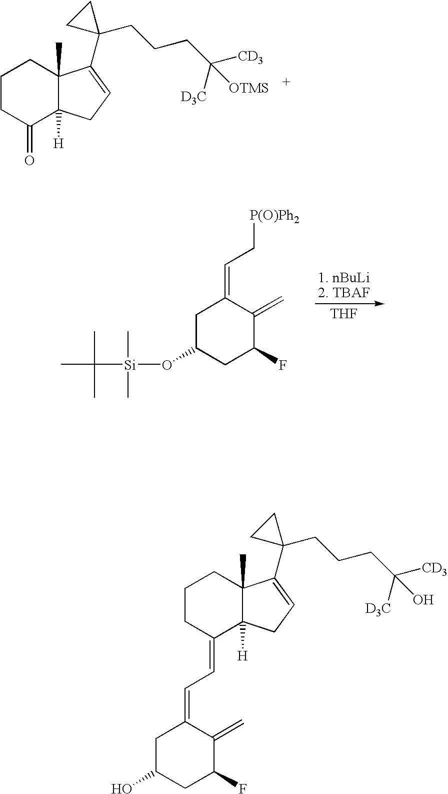

To a stirred solution of a (1S,5R)-1-((tert-butyldimethyl)silanyloxy)-3-[2-(diphenylphosphinoyl)-eth-(Z)-ylidene]-5-fluoro-2-methylene-cyclohexane (433 mg, 0.92 mmol) in tetrahydrofurane (7 mL) at −78° C. was added n-BuLi (0.58 mL, 0.93 mmol). The resulting mixture was stirred for 15 min and solution of (3aR,7aR)-7a-Methyl-1-{1-(5,5,5-trideutero-4-trideuteromethyl-4-trimethylsilanyloxy-pentyl)-cyclopropyl}-3a,4,5,6,7,7a-hexahydro-3H-inden-4-one (170 mg, 0.46 mmol, in tetrahydrofurane (2 mL) was added dropwise. The reaction mixture was stirred at −72° C. for 3.5 h diluted with hexane (25 mL) washed brine (20 mL) and dried over Na₂SO₄. The residue (580 mg) after evaporation of the solvent was purified by FC (15 g, 10% AcOEt in hexane) to give 1α-tert-Butyl-dimethyl-silanyloxy-3β-fluoro-25-trimethylsilanyloxy-16-ene-20-cyclopropyl-26,27-hexadeutero-cholecalciferol (260 mg, 042 mmol).

To the give 1α-tert-Butyl-dimethyl-silanyloxy-3β-fluoro-25-trimethylsilanyloxy-16-ene-20-cyclopropyl-26,27-hexadeutero-cholecalciferol (260 mg, 0.42 mmol) tetrabutylammonium fluoride (4 mL, 4 mmol, 1M solution in THF) was added, at room temperature. The mixture was stirred for 15 h. diluted with AcOEt (25 mL) and washed with water (5×20 mL), brine (20 mL) and dried over Na₂SO₄. The residue (260 mg) after evaporation of the solvent was purified by FC (10 g, 30%,50% AcOEt in hexane) to give the titled compound (140 mg, 0.32 mmol, 70%)

[α]³⁰_{D}=+30.0 c 0.30, EtOH

UV γmax (EtOH): 243 nm (ε 12254), 265 nm (ε 12144);

¹H NMR (CDCl₃): 6.40 (1H, d, J=11.3 Hz), 6.10 (1H, d, J=11.1 Hz), 5.39 (1H, s), 5.34 (1H, m), 5.13 (1H, dm, J=50 Hz), 5.11 (1H, s), 4.23 (1H, m), 2.80 (1H, m), 2.63 (1H, m), 2.38-1.08 (19H, m), 0.80 (3H, s),0.66-0.24 (4H, m);

¹³C NMR (CDCl₃): 156.92 (0), 143.06 (0, d, J=17 Hz), 142.78 (0), 131.49 (0), 125.48 (1), 124.71 (1), 117.16 (1), 114.67 (2, d, J=10 Hz), 91.47 (1, d, J=172 Hz), 70.83 (0), 66.58 (1, d, J=6 Hz), 59.55 (1), 50.35 (0), 45.00 (2), 43.80 (2), 40.79 (2, d, J=20 Hz), 38.20 (2), 35.68 (2), 29.15 (2), 28.74 (2), 23.64 (2), 22.32 (2), 20.79 (0), 17.96 (3), 12.81 (2), 10.41 (2);

| | | | |
|---|---|---|---|
| MS HRES | Calculated for C₂₈H₃₅FO₂D₆ | M+ | Na 457.3359 |
| | Observed | M+ | Na 457.3360 |

### Biological Examples

### Example 10

### Determination of Maximum Tolerated Dose (MTD)

The maximum tolerated dose of the vitamin D₃ compounds of the invention were determined in eight week-old female C57BL/6 mice (3 mice/group) dosed orally (0.1 ml/mouse) with various concentrations of Vitamin D₃ analogs daily for four days. Analogs were formulated in miglyol for a final concentration of 0.01, 0.03, 0.1, 0.3, 1, 3, 10, 30, 100 and 300 μg/kg when given at 0.1 ml/mouse p.o. daily. Blood for serum calcium assay was drawn by tail bleed on day five, the final day of the study. Serum calcium levels were determined using a colorimetric assay (Sigma Diagnostics, procedure no. 597). The highest dose of analog tolerated without inducing hypercalcemia (serum calcium>10.7 mg/dl) was taken as the maximum tolerated dose (MTD). Table 2 shows the relative MTD for compounds (1)-(9).

### Example 11

### Immunological Assay of Compounds (1)-(9)

Immature dendritic cells (DC) were prepared as described in Romani, N. et al. (Romani, N. et al. (1996) J. Immunol. Methyl. 196:137). IFN-γ production by allogeneic T cell activation in the mixed leukocyte response (MLR) was determined as described in Penna, G., et al., J Immunol., 164: 2405-2411 (2000).

Briefly, peripheral blood mononuclear cells (PBMC) were separated from buffy coats by Ficoll gradient and the same number (3×10⁵) of allogeneic PBMC from 2 different donors were co-cultured in 96-well flat-bottom plates. The vitamin D₃ compounds were added to each of the cultures. After 5 days, IFN-γ production in the MLR assay was measured by ELISA and the results expressed as amount (nM) of test compound required to induce 50% inhibition of IFN-γ production (IC₅₀). The results are summarized in Table 2.

TABLE 2

| | MTD (mice) | INF-γ |
|---|---|---|
| Compound | μg/kg | IC₅₀ pM |
| 1,25(OH)₂D₃ | 0.3 | 49.6 |
| 1,25-Dihydroxy-20-cyclopropyl-26,27-hexadeutero-19-nor- | 30 | 51.0 |
| cholecalciferol (1) | | |
| 1,3-Di-O-acetyl-1,25-dihydroxy-20-cyclopropyl-26,27- | 30 | 388.0 |
| hexadeutero-19-nor-cholecalciferol (2) | | |
| 1,25-Dihydroxy-20-cyclopropyl-26,27-hexadeutero- | 30 | 65.0 |
| cholecalciferol (3) | | |
| 1,3-Di-O-acetyl-1,25-dihydroxy-20-cyclopropyl-26,27- | 30 | 464.0 |
| hexadeutero-cholecalciferol (4) | | |
| 1α-Fluoro-25-hydroxy-20-cyclopropyl-26,27-hexadeutero- | 100 | >10000 |
| cholecalciferol (5) | | |
| 3-O-acetyl-1α-fluoro-25-hydroxy-20-cyclopropyl- | 30 | 747.0 |
| cholecalciferol (6) | | |
| 1,25-Dihydroxy-16-ene-20 cyclopropyl-26,27-hexadeutero- | 1 | 32.0 |
| 19-nor-cholecalciferol (7) | | |
| 1,25-Dihydroxy-16-ene-20-cyclopropyl-26,27-hexadeutero- | 10 | 63.0 |
| cholecalciferol (8) | | |
| 1α-Fluoro-25-hydroxy-16-ene-20-cyclopropyl-26,27- | 10 | 492.0 |
| hexadeutero-cholecalciferol (9) | | |

### Example 12

### Proliferation Assay using Bladder Cancer Cell Lines

Bladder cancer cell lines (T24, RT112, HT1376 and RT4 are human bladder cancer cell lines; NHEK are normal human keratinocytes) were obtained from the European Collection of Cell Cultures (Salisbury, UK). Cells were plated at 3×103 per well, in flat bottomed 96-well plates in 100 μl of DMEM medium containing: 5% Fetal Clone i, 50 μg/l gentamicin, 1 mM sodium pyruvate and 1% non-essential amino acids. After culturing for 24 h at 37° C. in 5% CO2, to allow cells to adhere to the plates, VDR ligands (compounds (1)-(9)) were added at concentrations ranging from 100 μM to 0.3 μM in 100 μl of above-mentioned complete medium. After a further 72 h of culture, cell proliferation was measured using a fluorescence-based proliferation assay kit. (CyQuant Cell Proliferation Assay Kit, Molecular Probes, Eugene, Oreg., USA). The IC50 was calculated from the regression curve of the titration data. The results are shown in Table 3.

TABLE 3

| | T24 | RT112 | HT 1376 | RT4 | NHEK |
|---|---|---|---|---|---|
| Compound | (μM) | (μM) | (μM) | (μM) | (μM) |
| 1,25(OH)₂D₃ | 54.6 | 19/28.7 | 50 | 45/26 | 4.5 |
| 1,25-Dihydroxy-20-cyclopropyl-26,27- | — | >30 | — | 11.7 | 5.6 |
| hexadeutero-19-nor-cholecalciferol (1) | | | | | |
| 1,3-Di-O-acetyl-1,25-dihydroxy-20- | — | 16.3 | — | 3.1 | 7.2 |
| cyclopropyl-26,27-hexadeutero-19-nor- | | | | | |
| cholecalciferol (2) | | | | | |
| 1,25-Dihydroxy-20-cyclopropyl-26,27- | — | 19.7 | — | 2.9 | 6.6 |
| hexadeutero-cholecalciferol (3) | | | | | |
| 1,3-Di-O-acetyl-1,25-dihydroxy-20- | — | 18.7 | — | 3 | 5.6 |
| cyclopropyl-26,27-hexadeutero-cholecalciferol | | | | | |
| (4) | | | | | |
| 1α-Fluoro-25-hydroxy-20-cyclopropyl-26,27- | — | 15.7 | — | 3.1 | 4.7 |
| hexadeutero-cholecalciferol (5) | | | | | |
| 3-O-acetyl-1α-fluoro-25-hydroxy-20- | — | 19.7 | — | 4.5 | 5.6 |
| cyclopropyl-cholecalciferol (6) | | | | | |
| 1,25-Dihydroxy-16-ene-20 cyclopropyl-26,27- | — | >30 | — | 8.9 | >10 |
| hexadeutero-19-nor-cholecalciferol (7) | | | | | |
| 1,25-Dihydroxy-16-ene-20-cyclopropyl-26,27- | — | 30 | — | 2.4 | 7.0 |
| hexadeutero-cholecalciferol (8) | | | | | |
| 1α-Fluoro-25-hydroxy-16-ene-20-cyclopropyl- | — | 30 | — | 4.3 | 7.0 |
| 26,27-hexadeutero-cholecalciferol (9) | | | | | |

### Example 13

### The Activity of Calcitriol and Vitamin D₃ Analogues on the Growth and Function of Bladder Cells

The Inventors' finding that calcitriol and Vitamin D₃ analogues can have an effect on the growth and function of bladder cells has been proven in in vitro models by culturing human stromal bladder cells. The Inventors confirm the presence of vitamin D receptors (VDRs), as previously reported in the literature, on these cells.

In these models, calcitriol (the activated form of vitamin D₃) and other vitamin D₃ analogues have been shown to be effective in inhibiting the basal growth of bladder cells. This activity, never reported before, is dose dependent with an IC₅₀ of 9.8±7×10⁻¹⁵ for calcitriol (1,25-dihydroxycholecalciferol) (on basal cells).

A similar investigation is performed on a number of other vitamin D compounds and the results (expressed as -Log IC₅₀) are shown in the table below. Data in the table refers to inhibitors effect of the compound on basal human bladder cell growth in cells which are not stimulated with testosterone or (in one case) are stimulated. The maximum tolerated dose (MTD) in rats is also listed for each compound.

### Example 14

### Renin mRNA Inhibition in Murine Juxtaglomerular Cell Line As4.1

As4.1 cells (80% subconfluent) were treated in complete medium with compounds of the invention at 10⁻⁸,10⁻⁹ and 10⁻¹⁰ M for 24 h. Total RNA was extracted using RNeasy Mini kit (Qiagen); treated with DNase I (Qiagen) and Reverse Transcription Reagent (Applied Biosystems) with Random Examers were used for reverse transcription according to the manufacturers' instructions. Real Time PCR analysis was performed in multiplex using commercially available β-actin VIC-conjugated probe (cat. n. 4352341E, Applied Biosystems) and custom designed mRENIN FAM-conjugated probe (Assay by Design, Applied Biosystems; Forward: AGGCCTTCCTTGACCAATCTTAC; Reverse: GCTGAACCCGTGTCAAAGATG; Probe: FAM-ACCAACTACCTGAATACCGAGT-MGB). Reactions were performed in a 25 μL volume containing 12.5 μl 12× Master Mix (Applied Biosystems), 10 ng/reaction/well cDNA, and 2.5 μM each gene-specific primer. An ABI PRISM 7700 analyzer (Applied Biosystems) was used at 50° C. for 2 minutes and 95° C. for 10 minutes, followed by 40 cycles at 95° C. for 15 seconds and 60° C. for 1 minute. Cycle threshold (Ct) values were exported onto Excel worksheets for analysis. Test cDNA results were normalized versus mouse β-actin housekeeping gene. Fold differences in gene expression between the subtracted versus the unsubtracted populations were expressed using the method of 2^{-ΔΔCt}. (Table 4)

TABLE 4

| | Renin mRNA |
|---|---|
| | inh.As4.1/wt; |
| Compound | IC50 pM |
| 1,25-Dihydroxy-20-cyclopropyl-26,27- | 154700.8 |
| hexadeutero-19-nor-cholecalciferol (1) | |
| 1,3-Di-O-acetyl-1,25-dihydroxy-20-cyclopropyl- | 101731.7 |
| 26,27-hexadeutero-19-nor-cholecalciferol (2) | |
| 1,25-Dihydroxy-20-cyclopropyl-26,27- | 42878.0 |
| hexadeutero-cholecalciferol (3) | |
| 1,3-Di-O-acetyl-1,25-dihydroxy-20-cyclopropyl- | 274031.7 |
| 26,27-hexadeutero-cholecalciferol (4) | |
| 1α-Fluoro-25-hydroxy-20-cyclopropyl-26,27- | 100000000.0 |
| hexadeutero-cholecalciferol (5) | |
| 3-O-acetyl-1α-fluoro-25-hydroxy-20- | 4475.7 |
| cyclopropyl-cholecalciferol (6) | |
| 1,25-Dihydroxy-16-ene-20 cyclopropyl-26,27- | 7356.2 |
| hexadeutero-19-nor-cholecalciferol (7) | |
| 1,25-Dihydroxy-16-ene-20-cyclopropyl-26,27- | 4890.0 |
| hexadeutero-cholecalciferol (8) | |
| 1α-Fluoro-25-hydroxy-16-ene-20-cyclopropyl- | 12790.1 |
| 26,27-hexadeutero-cholecalciferol (9) | |

### Example 15

Soft Gelatin Capsule Formulation I

| Item | Ingredients | mg/Capsule |
|---|---|---|
| 1 | Compound (1) from Example 1 | 10.001-0.02 |
| 2 | Butylated Hydroxytoluene (BHT) | 0.016 |
| 3 | Butylated Hydroxyanisole (BHA) | 0.016 |
| 4 | Miglyol 812 qs. | 160.0 |

### Manufacturing Procedure:

1. BHT and BHA is suspended in Miglyol 812 and warmed to about 50° C. with stirring, until dissolved.

2. 1,25-Dihydroxy-20-cyclopropyl-26,27-hexadeutero-19-nor-cholecalciferol is dissolved in the solution from step 1 at 50° C.

3. The solution from Step 2 is cooled at room temperature.

4. The solution from Step 3 is filled into soft gelatin capsules.

Note: All manufacturing steps are performed under a nitrogen atmosphere and protected from light.

### Example 16

Soft Gelatin Capsule Formulation II

| Item | Ingredients | mg/Capsule |
|---|---|---|
| 1 | Compound(1)from Example 1 | 10.001-0.02 |
| 2 | di-.alpha.-Tocopherol | 0.016 |
| 3 | Miglyol 812 qs. | 160.0 |

### Manufacturing Procedure:

1. Di-α-Tocopherol is suspended in Miglyol 812 and warmed to about 50° C. with stirring, until dissolved.

2. 1,25-Dihydroxy-20-cyclopropyl-26,27-hexadeutero-19-nor-cholecalciferol is dissolved in the solution from step 1 at 50° C.

3. The solution from Step 2 is cooled at room temperature.

4. The solution from Step 3 is filled into soft gelatin capsules.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A vitamin D₃ compound of formula I: 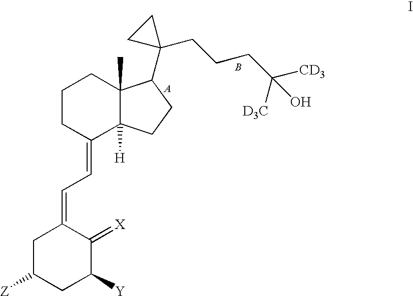
wherein:
A is single or double bond;
B is a single, double, or triple bond;
X is H₂ or CH₂;
Y is hydroxyl, OC(O)C₁-C₄ alkyl, OC(O)hydroxyalkyl, OC(O)haloalkyl; or halogen;
Z is hydroxyl, OC(O)C₁-C₄ alkyl, OC(O)hydroxyalkyl, or OC(O)haloalkyl; and
pharmaceutically acceptable esters, salts, and prodrugs thereof.

2. The compound of claim 1, wherein Y is hydroxyl.

3. The compound of claim 1, wherein Y is halogen.

4. The compound of claim 3, wherein Y is F.

5. The compound of claim 1, wherein Y is OC(O)C₁-C₄ alkyl.

6. The compound of claim 5, wherein Y is OC(O)CH₃.

7. The compound of claim 1, wherein Z is hydroxyl.

8. The compound of claim 1, wherein A is a single bond.

9. The compound of claim 1, wherein A is a double bond.

10. The compound of claim 1, wherein B is a single bond.

11. The compound of claim 1, wherein B is a double bond.

12. The compound of claim 1, wherein B is a triple bond.

13. The compound of claim 1, wherein X is CH₂.

14. The compound of claim 1, wherein X is H₂.

15. The compound of claim 1, wherein said compound is 1,25-Dihydroxy-20-cyclopropyl-26,27-hexadeutero-19-nor-cholecalciferol:

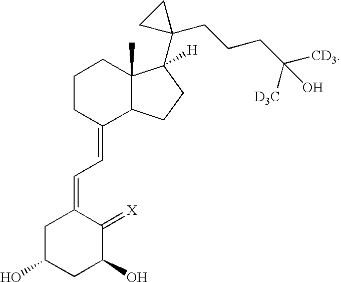

16. The compound of claim 1, wherein said compound is 1,3-Di-O-acetyl-1,25-dihydroxy-20-cyclopropyl-26,27-hexadeutero-19-nor-cholecalciferol:

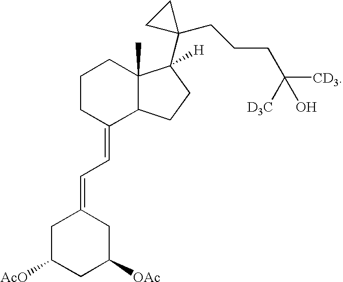

17. The compound of claim 1, wherein said compound is 1,25-Dihydroxy-20-cyclopropyl-26,27-hexadeutero-cholecalciferol:

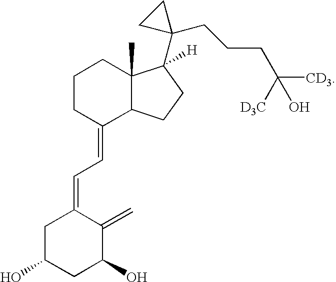

18. The compound of claim 1, wherein said compound is 1,3-Di-O-acetyl-1,25-dihydroxy-20-cyclopropyl-26,27-hexadeutero-cholecalciferol:

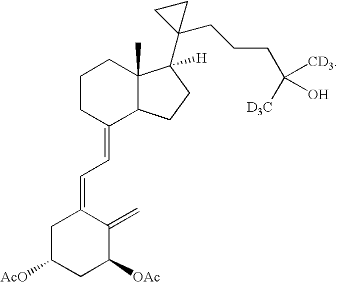

19. The compound of claim 1, wherein said compound is 1α-Fluoro-25-hydroxy-20-cyclopropyl-26,27-hexadeutero-cholecalciferol:

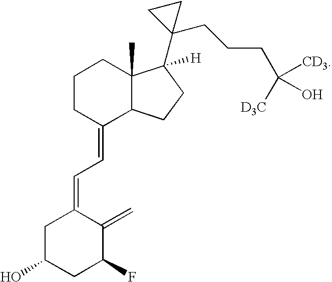

20. The compound of claim 1, wherein said compound is 3-O-acetyl-1α-fluoro-25-hydroxy-20-cyclopropyl-cholecalciferol:

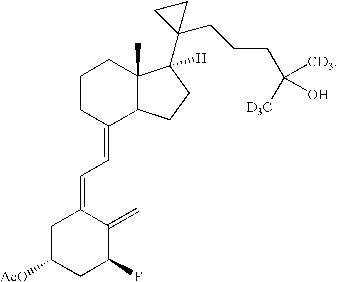

21. The compound of claim 1, wherein said compound is 1,25-Dihydroxy-16-ene-20 cyclopropyl-26,27-hexadeutero-19-nor-cholecalciferol:

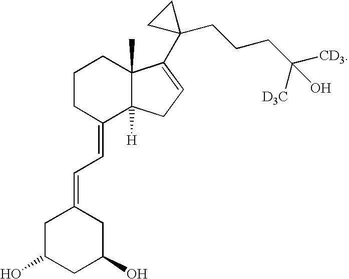

22. The compound of claim 1, wherein said compound is 1,25-Dihydroxy-16-ene-20-cyclopropyl-26,27-hexadeutero-cholecalciferol:

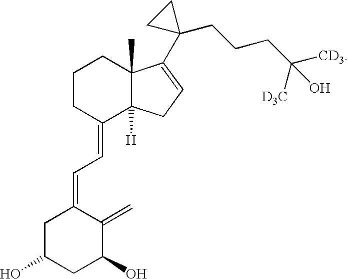

23. The compound of claim 1, wherein said compound is 1α-Fluoro-25-hydroxy-16-ene-20-cyclopropyl-26,27-hexadeutero-cholecalciferol:

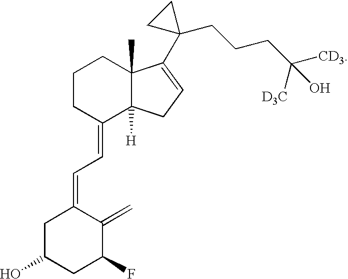

24. A method for treating a subject for a vitamin D₃ associated state, comprising administering to a subject in need thereof an effective amount of a vitamin D₃ compound of claim 1, such that said subject is treated for said vitamin D₃ associated state.

25. (canceled)

26. The method of claim 24, further comprising identifying a subject in need of treatment for a vitamin D₃ associated state.

27. The method of claim 24, wherein said vitamin D₃ associated state is disorder selected from the group consisting of an ILT3-associated disorder, a disorder characterized by an aberrant activity of a vitamin D₃-responsive cell.

28. The method of claim 27, wherein said ILT3-associated disorder is an immune disorder or an autoimmune disorder.

29. The method of claim 28, wherein said immune disorder is an autoimmune disorder selected from the group consisting of type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitis, uveoretinitis, leukocyte adhesion deficiency, rheumatoid arthritis, rheumatic fever, Reiter's syndrome, psoriatic arthritis, progressive systemic sclerosis, primary biliary cirrhosis, pemphigus, pemphigoid, necrotizing vasculitis, myasthenia gravis multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease.

30-32. (canceled)

33. The method of claim 24, wherein said disorder characterized by an aberrant activity of a vitamin D₃-responsive cell is selected from the group consisting of a disorder characterized by an aberrant activity of a hyperproliferative skin cell, a disorder characterized by an aberrant activity of an endocrine cell, a disorder characterized by an aberrant activity of a bone cell, a disorder characterized by an aberrant activity of a vitamin D₃-responsive smooth muscle cell cirrhosis or chronic renal disease, hypertension, benign prostate hypertrophy, neoplastic disease, neuronal loss, uveitis, interstitial cystitis.

34. (canceled)

35. The method of claim 33, wherein said disorder characterized by an aberrant activity of a hyperproliferative skin cell is selected from the group consisting of psoriasis, basal cell carcinoma and keratosis.

36. (canceled)

37. (canceled)

38. The method of claim 33, wherein said disorder characterized by an aberrant activity of an endocrine cell is secondary hyperparathyroidism.

39. (canceled)

40. (canceled)

41. The method of claim 33, wherein said disorder characterized by an aberrant activity of a bone cell is selected from the group consisting of osteoporosis, osteodystrophy, senile osteoporosis, osteomalacia, rickets, osteitis fibrosa cystica, and renal osteodystrophy.

42-45. (canceled)

46. The method of claim 33, wherein said disorder is hypertension and the compound is 1,25-Dihydroxy-20-cyclopropyl-26,27-hexadeutero-19-nor-cholecalciferol: 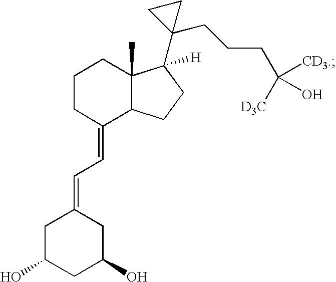
1,3-Di-O-acetyl-1,25-dihydroxy-20-cyclopropyl-26,27-hexadeutero-19-nor-cholecalciferol: 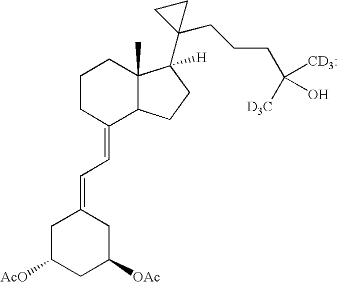
1,25-Dihydroxy-20-cyclopropyl-26,27-hexadeutero-cholecalciferol: 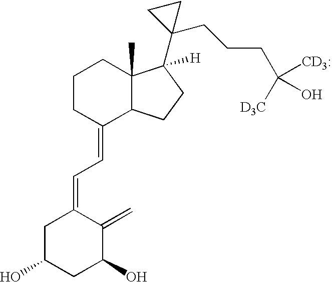
1,3-Di-O-acetyl-1,25-dihydroxy-20-cyclopropyl-26,27-hexadeutero-cholecalciferol: 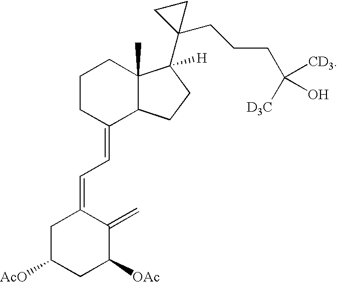
1α-Fluoro-25-hydroxy-20-cyclopropyl-26,27-hexadeutero-cholecalciferol: 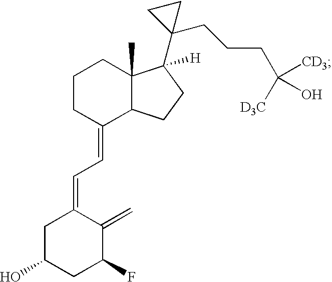
3-O-acetyl-1α-fluoro-25-hydroxy-20-cyclopropyl-cholecalciferol: 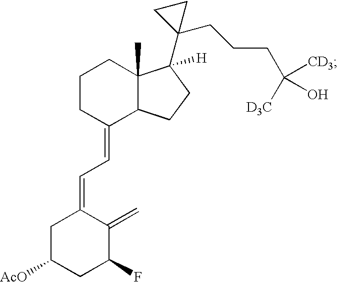
1,25-Dihydroxy-16-ene-20 cyclopropyl-26,27-hexadeutero-19-nor-cholecalciferol: 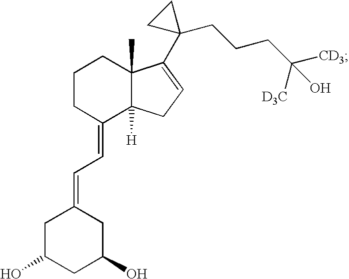
1,25-Dihydroxy-16-ene-20-cyclopropyl-26,27-hexadeutero-cholecalciferol: 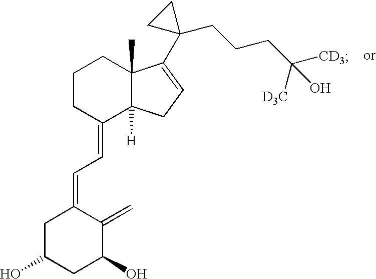
1α-Fluoro-25-hydroxy-16-ene-20-cyclopropyl-26,27-hexadeutero-cholecalciferol:

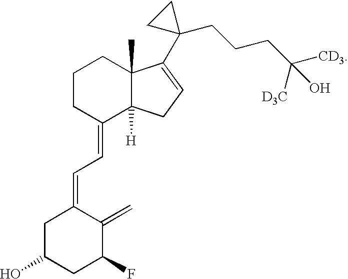

47-56. (canceled)

57. The method of claim 33, wherein the neoplastic disease is selected from the group consisting of leukemia, lymphoma, melanoma, osteosarcoma, colon cancer, rectal cancer, prostate cancer, bladder cancer, and malignant tumors of the lung, breast, gastrointestinal tract, and genitourinary tract.

58. (canceled)

59. (canceled)

60. The method of claim 33, wherein the disorder comprises neuronal loss and is selected from the group consisting of Alzheimer's Disease, Pick's Disease, Parkinson's Disease, Vascular Disease, Huntington's Disease, and Age-Associated Memory Impairment.

61-63. (canceled)

64. The method of claim 33, wherein the disorder is characterized by an aberrant activity of a vitamin D₃-responsive smooth muscle cell and is selected from the group consisting of uterine myomas, hyperproliferative vascular disease and arterial hypertension.

65. (canceled)

66. (canceled)

67. A method of ameliorating a deregulation of calcium and phosphate metabolism, comprising administering to a subject in need thereof a therapeutically effective amount of a compound of claim 1, so as to ameliorate the deregulation of the calcium and phosphate metabolism in said subject.

68. (canceled)

69. A method of modulating the expression of an immunoglobulin-like transcript 3 (ILT3) surface molecule in a cell, comprising contacting said cell with a compound of claim 1, in an amount effective to modulate the expression of an immunoglobulin-like transcript 3 (ILT3) surface molecule in said cell.

70. (canceled)

71. A method of treating an ILT3-associated disorder in a subject, comprising administering to a subject in need thereof a compound of claim 1, in an amount effective to modulate the expression of an ILT3 surface molecule, thereby treating said ILT3-associated disorder in said subject.

72-74. (canceled)

75. A method of inducing immunological tolerance in a subject, comprising administering to a subject in need thereof a compound of claim 1, in an amount effective to modulate the expression of an ILT3 surface molecule, thereby inducing immunological tolerance in said subject.

76. (canceled)

77. (canceled)

78. A method of inhibiting transplant rejection in a subject comprising administering to a subject in need thereof a compound claim 1, in an amount effective to modulate the expression of an ILT3 surface molecule, thereby inhibiting transplant rejection in said subject.

79-81. (canceled)

82. A method for modulating immunosuppressive activity by an antigen-presenting cell, comprising contacting an antigen-presenting cell with a compound of claim 1, in an amount effective to modulate ILT3 surface molecule expression, thereby modulating said immunosuppressive activity by said antigen-presenting cell.

83. (canceled)

84. A method for preventing or treating bladder dysfunction in a subject comprising administering to a subject in need thereof an effective amount of a compound of claim 1, thereby to prevent or treat bladder dysfunction in said subject.

85-95. (canceled)

96. The method of claim 24, wherein the subject is a mammal.

97. The method of claim 96, wherein the subject is human.

98. The method of claim 24, wherein said compound is administered orally, intravenously, topically or parenterally.

99-101. (canceled)

102. The method of claim 24, wherein said compound is administered at a concentration of 0.001 μg-100 μg/kg of body weight.

103. A pharmaceutical composition, comprising a compound of any one of claim 1, and a pharmaceutically acceptable diluent or carrier.

104. The pharmaceutical composition of claim 103, wherein said compound is present in an amount effective to treat a vitamin D₃ associated state.

105-108. (canceled)

109. A packaged formulation for use in the treatment of a vitamin D₃ associated state, comprising a pharmaceutical composition comprising a compound of claim 1, and instructions for use in the treatment of a vitamin D₃ associated state.

110-112. (canceled)
